(19) United States
(12) Patent Application Publication
(10) Pub. No.: US 2006/0041223 A1
(43) Pub. Date: Feb. 23, 2006

(54) All-in-one interface for programmable implantable medical device

(75) Inventors: Wende L. Dewing, Edina, MN (US); Dale R. Ulbrich, Maple Grove, MN (US); Pavankumar Dadlani, Minneapolis, MN (US)

Correspondence Address: IPLM GROUP, P.A., POST OFFICE BOX 18455, MINNEAPOLIS, MN, 55418, US

(73) Assignee: Medtronic, Inc., Minneapolis, MN (US)

(21) Appl. No.: 11/206,681

(22) Filed: Aug. 18, 2005

Related U.S. Application Data
(60) Provisional application No. 60/602,489, filed on Aug. 18, 2004.

(51) Int. Cl. A61M 31/00

(52) U.S. Cl. 604/93.01

(57) ABSTRACT

A system capable of delivering a therapeutic output to a patient. An implantable medical device is capable of delivering the therapeutic output to the patient. A controller, programmable by a medical professional, is operatively coupled to the implantable medical device, to specify, at least in part, the therapeutic output to be delivered to the patient. The controller is operable to specify the therapeutic output through specification of a plurality of tasks. The controller has an interface providing a first screen presenting the medical professional with at least some of the plurality of tasks on the first screen with the first screen divided into a plurality of task areas with each of the at least some of the plurality of tasks associated with a different one of the plurality of task areas.

### RELATED APPLICATIONS

This application claims priority to U.S. Provisional Patent Application Ser. No. 60/602,489, Wende L. Dewing, Dale R. Ulbrich and Pavankumar Dadlani, filed Aug. 18, 2004, entitled Interface For Programmable Implantable Medical Device.

### FIELD OF THE INVENTION

This invention relates to implantable medical device systems and, in particular, interfaces for implantable medical device systems that are programmable by a medical professional.

### BACKGROUND OF THE INVENTION

Implantable medical devices for producing a therapeutic result in a patient are well known. Examples of such implantable medical devices include implantable drug infusion pumps, implantable neurostimulators, implantable cardioverters, implantable cardiac pacemakers, implantable defibrillators and cochlear implants. Of course, it is recognized that other implantable medical devices are envisioned.

These devices are intended to provide a patient with a therapeutic output to alleviate or assist with a variety of conditions. Typically such devices are implanted in a patient and provide a therapeutic output under specified conditions on a recurring basis.

One type implantable medical device is a drug infusion device which can deliver a medication, typically fluid medication, to a patient at a selected site. A drug infusion device may be implanted at a location in the body of a patient and deliver a fluid medication through a catheter to a selected delivery site in the body. Examples of such devices are described in U.S. Pat. No. 5,782,798, Rise, entitled Techniques For Treating Eating Disorders By Brain Stimulation and Drug Infusion; U.S. Pat. No. 5,814,014, Elsberry et al, Techniques of Treating Neurodegnerative Disorders by Brain Infusion, each assigned to Medtronic, Inc., Minneapolis, Minn.

Another type of implantable medical device is an electrical stimulation device. An electrical nerve stimulator can also be implanted in the body of a patient and can stimulate selected nerves in the body in accordance with a specified routine. The electrical nerve stimulator may be implanted at a location in the body and deliver electrical stimulation pulses through a lead or leads to a stimulus site. Examples of such an implantable electrical stimulation device are Medtronic's Itrel®3 and Soletra™ neurostimulators.

It is desirable to be able non-invasively program an implanted medical device, such as a drug infusion device or an electrical stimulation device, in order to change to therapeutic regimen without incurring unnecessary trauma to the patient. An example of such a device is described in U.S. Pat. No. 4,692,147, Duggan, Drug Administration Device, assigned to Medtronic, Inc., Minneapolis, Minn., which can be non-invasively programmed to change both the dosage amount and the dosage interval. Verification of the received dosage and interval commands is achieved by means of an audio transducer which is attached to the device case.

The implantable drug administration device described in Duggan allows a medical professional to program to the delivery rate of a drug contained in the reservoir of the device over a specified interval. The process, however, to achieve an even reasonably complex dosing regimen is laborious and time consuming. Each interval must be specified and the particular delivery rate must be individually programmed. For all but the simplest of dosing regimens, this system is not only laborious and takes too long to program but also prone to error due to the painstaking programming steps which must be accomplished.

Non-invasively programmable implantable medical devices are typically programmed using an external programming device, sometimes known as a controller, which can communicate with the implanted medical device through well known techniques such as telemetry. An external controller, or programmer, can be used by a medical professional, for example, to change to therapeutic regimen by increasing or decreasing the amount or timing of fluid medication delivered or by increasing or decreasing the intensity or timing or characteristic of an electrical stimulation signal. Typically, a medical professional interfaces with the external controller or programmer to set various parameters associated with the implantable medical device and then transmits, or downloads, those parameters to the implanted medical device. The external device may also record other information important to the deliver of the therapeutic output although not actually downloaded to the implanted medical, e.g., patient information, implanted device information such as model, volume, implant location, length of catheter or lead, etc.

### BRIEF SUMMARY OF THE INVENTION

The external controller or programmer typically has an interface which allows the medical professional to effectively utilize the external controller and efficiently utilize its features. The various aspects of the present invention provide an interface for a controller or programmer of an implantable medical device which allow for a medical professional to efficiently and effectively utilize the various features of the implantable medical device. The proper interface with the medical professional can allow the medical professional to reduce errors, increase productivity, increase the medical professional's understanding of the implantable medical device system and increase the medical professional's confidence with the implantable medical device system.

The interfaces associated with the present invention provides the medical professional with an interface that is task oriented and logical in sequence. The interfaces typically are easy to manipulate and require fewer steps, i.e., entries, clicks, drags and screens, than previous interfaces. In at least some embodiments, the interfaces are presented in clinical terms which the medical professional understands rather than in engineering which the implantable medical device designers understand but with which the medical professional may be unfamiliar.

In an embodiment, the present invention provides a system capable of delivering a therapeutic output to a patient. An implantable medical device is capable of delivering the therapeutic output to the patient. A controller, programmable by a medical professional, is operatively coupled to the implantable medical device, to specify, at least in part, the therapeutic output to be delivered to the patient. The controller is operable to specify the therapeutic output through specification of a plurality of tasks. The controller has an interface providing a first screen presenting the medical professional with at least some of the plurality of tasks on the first screen with the first screen divided into a plurality of task areas with each of the at least some of the plurality of tasks associated with a different one of the plurality of task areas.

In an alternative embodiment, the present invention provides a controller for an implantable medical device capable of delivering a therapeutic output to a patient. A control module, is operatively coupled to the implantable medical device, being programmable by a medical professional to specify, at least in part, the therapeutic output to be delivered to the patient. The control module is operable to specify the therapeutic output through specification of a plurality of tasks. The control module has an interface providing a first screen presenting the medical professional with at least some of the plurality of tasks on the first screen with the first screen divided into a plurality of task areas with each of the at least some of the plurality of tasks associated with a different one of the plurality of task areas.

In an embodiment, the interface presents a second screen associated with a particular one of the plurality of tasks upon selection of the task by the medical professional from the first screen.

In an embodiment, the interface represents the first screen upon completion by the medical professional of the particular one of the plurality of tasks.

In an embodiment, the first screen distinctly identifies the tasks already selected by the medical professional.

In an embodiment, the first screen distinctly identifies the tasks already completed by the medical professional.

In an alternative embodiment, the present invention provides a method of controlling an implantable medical device capable of delivering a therapeutic output to a patient, the implantable medical device being programmable by a medical professional to specify through a plurality of tasks, at least in part, the therapeutic output to be delivered to the patient. An interface is presented providing a first screen presenting the medical professional with at least some of the plurality of tasks on the first screen with the first screen divided into a plurality of task areas with each of the at least some of the plurality of tasks associated with a different one of the plurality of task areas. A second screen is presented associated with a particular one of the plurality of tasks upon selection of the task by the medical professional from the first screen.

In an embodiment, the interface represents the first screen upon completion by the medical professional of the particular one of the plurality of tasks.

In an embodiment, the first screen distinctly identifies the tasks already selected by the medical professional.

### BRIEF DESCRIPTION OF THE DRAWINGS

FIG. 1 is a schematic view of an implantable medical device system of the present invention having an implantable medical device, in this case a drug infusion device, implanted within a patient's body;

FIG. 2 is a block diagram of the system of FIG. 1 having an implantable drug infusion device and an external programmer;

FIG. 3 illustrates an introductory screen shot;

FIG. 4 is a warning screen shot indicative of low batteries in the programmer;

FIG. 5 is an informational screen shot providing instructions for beginning a telemetry session;

FIG. 6 is an informational screen shot indicating that the external programmer is searching for the implanted medical device;

FIG. 7 is an informational screen shot communicating an inability to establish telemetry;

FIG. 8 is an informational screen shot communicating telemetry failure;

FIG. 9 is an informational screen shot communicating application loading for telemetry in progress;

FIG. 10 is an informational screen shot communicating pump interrogation in progress during telemetry;

FIG. 11 is an informational screen shot communicating uploading of notes during telemetry;

FIG. 12 illustrates a screen shot of a task based flow interface listing tasks pertinent to an implant/surgical procedure;

FIG. 13 illustrates a screen shot of a task based flow interface listing tasks pertinent to a refill procedure;

FIG. 14 illustrates a screen shot of a task based flow interface listing tasks pertinent to a troubleshooting procedure;

FIG. 15 illustrates an alternative embodiment of a task based flow introductory screen shot with active alarms;

FIG. 16 illustrates an alternative embodiment of a task based flow introductory screen shot with alarms silenced;

FIG. 17 illustrates a screen shot of a refill only procedure selection;

FIG. 18 illustrates a screen shot of optional steps to be selected for the refill only procedure;

FIG. 19 illustrates a screen shot of steps taken during the refill only procedure;

FIG. 20 illustrates a screen shot associated with an initial status of the drug infusion device associated with the “implant/surgical” procedure;

FIG. 21 illustrates a screen shot associated with an initial status of the drug infusion device associated with the “refill” procedure;

FIG. 22 illustrates a screen shot associated with an initial status of the drug infusion device associated with the “troubleshooting” procedure;

FIG. 23 illustrates a screen shot of the second embodiment of the pump information task area in which information about the catheter may be provided;

FIG. 24 illustrates a screen shot of the second embodiment of the pump information task area in which additional information concerning the pump installation may be provided;

FIG. 25 illustrates a screen shot of a third embodiment of the pump information task area in which detailed pump information may be provided;

FIG. 26 illustrates a screen shot showing an “all in one” interface of an implanted drug infusion device containing multiple drugs;

FIG. 27 illustrates a screen shot showing an “all in one” interface of an implanted drug infusion device containing a single drug with no boluses set;

FIG. 28 illustrates a screen shot showing an “all in one” interface of an implanted drug infusion device either before implantation or following implantation but before initial parameters have been input.

FIG. 29 illustrates a screen shot showing an “all in one” interface of an implanted drug infusion device with the “pump” task area and the “refill” task area identified with a distinctive background;

FIG. 30 illustrates a screen shot showing an initial status screen with warnings;

FIG. 31 illustrates a screen shot showing an initial status screen with device information;

FIG. 32 illustrates a screen shot showing details on the contents of a drug contained in the reservoir of an implanted drug infusion device;

FIG. 33 illustrates a screen shot of a patient information task area in which detailed patient information may be provided;

FIG. 34 illustrates a screen shot of a patient information task area information that has been modified;

FIG. 35 illustrates a screen shot of a patient information task area in which a note concerning the patient may be provided;

FIG. 36 illustrates a screen shot of an embodiment of the pump information task area in which detailed pump information may be provided;

FIG. 37 illustrates a screen shot of an embodiment of the pump information task area in which catheter information may be provided;

FIG. 38 illustrates a screen shot of an embodiment of the pump information task area in which further information about the catheter may be provided;

FIG. 39 illustrates a screen shot showing the entry of numerical values;

FIG. 40 illustrates a screen shot of an embodiment of detailed information about the catheter;

FIG. 41 illustrates a screen shot of an embodiment of detailed information about the catheter;

FIG. 42 illustrates a screen shot showing information associated a volume in the reservoir;

FIG. 43 illustrates a screen shot showing information on a previous reservoir volume and entry of a new reservoir volume;

FIG. 44 illustrates a screen shot showing the newly entered reservoir volume information;

FIG. 45 illustrates a screen shot showing the contents of water in the reservoir;

FIG. 46 illustrates a screen shot showing entry of one drug;

FIG. 47 illustrates a screen shot showing entry of three drugs in the reservoir;

FIG. 48 illustrates a screen shot showing a list of drugs to be added;

FIG. 49 illustrates a screen shot showing a listed drug being selected;

FIG. 50 illustrates a screen shot ready for the addition of a drug to the list;

FIG. 51 illustrates a screen shot showing the selection of a dosing unit for the added drug;

FIG. 52 illustrates a screen shot showing an added drug being selected;

FIG. 53 illustrates a screen shot showing a concentration being removed;

FIG. 54 illustrates a screen shot showing a concentration being selected;

FIG. 55 illustrates a screen shot showing numerical entry of a new concentration;

FIG. 56 illustrates a screen shot showing a new concentration having been numerically entered;

FIG. 57 illustrates a screen shot showing selection of a single bolus drug infusion program;

FIG. 58 illustrates a screen shot showing the absence of a single bolus drug infusion program;

FIG. 59 illustrates a screen shot showing entry of a single bolus drug infusion program;

FIG. 60 illustrates a screen shot showing numerical entry of a bolus dose;

FIG. 61 illustrates a screen shot showing an out of range bolus dose warning;

FIG. 62 illustrates a screen shot showing an entered bolus dose;

FIG. 63 illustrates a screen shot showing a summary of a single bolus drug infusion program;

FIG. 64 illustrates a screen shot showing introduction of a prime bolus;

FIG. 65 illustrates a screen shot showing selection of prime bolus components;

FIG. 66 illustrates a screen shot showing selection prime bolus components;

FIG. 67 illustrates a screen shot showing selection of prime bolus components using pump tubing only;

FIG. 68 illustrates a screen shot showing pump tubing entry;

FIG. 69 illustrates a screen shot showing pump tubing selection and data entry;

FIG. 70 illustrates a screen shot showing a summary of a prime bolus drug infusion program;

FIG. 71 illustrates a screen shot showing a prime bolus calculation;

FIG. 72 illustrates a screen shot showing an alternative prime bolus calculation;

FIG. 73 illustrates a screen shot showing introducing a bridge bolus;

FIG. 74 illustrates a screen shot allowing entry of a bridge bolus;

FIG. 75 illustrates a screen shot showing a bridge bolus dosage amount;

FIG. 76 illustrates a screen shot showing a summary of a bridge bolus drug infusion program;

FIG. 77 illustrates a screen shot showing a bridge bolus calculation;

FIG. 78 illustrates a screen shot showing selection of a drug infusion program type;

FIG. 79 illustrates a screen shot showing manual entry of a simple drug infusion program;

FIG. 80 illustrates a screen shot showing graphical entry of a simple drug infusion program;

FIG. 81 illustrates a screen shot showing selection of a day-night drug infusion program;

FIG. 82 illustrates a screen shot showing manual entry of a day-night drug infusion program;

FIG. 83 illustrates a screen shot showing graphical entry of a day-night drug infusion program;

FIG. 84 illustrates a screen shot showing selection of a periodic drug infusion program;

FIG. 85 illustrates a screen shot showing manual entry of a periodic drug infusion program;

FIG. 86 illustrates a screen shot showing graphical entry of a periodic drug infusion program;

FIG. 87 illustrates a screen shot showing selection of a flexible drug infusion program;

FIG. 88 illustrates a screen shot showing manual entry of a flexible drug infusion program;

FIG. 89 illustrates a screen shot showing an alternative embodiment of manual entry of a flexible drug infusion program;

FIG. 90 illustrates a screen shot showing another embodiment of manual entry of a flexible drug infusion program;

FIG. 91 illustrates a screen shot showing another embodiment of manual entry of a flexible drug infusion program;

FIG. 92 illustrates a screen shot showing graphical entry of a flexible drug infusion program;

FIG. 93 illustrates a screen shot showing selection of days of programming to copy;

FIG. 94 illustrates a screen shot showing Tuesday as a selected day of programming to copy;

FIG. 95 illustrates a screen shot showing Monday through Thursday as selected days of programming to copy;

FIG. 96 illustrates a screen shot showing an introduction to a patient controlled activation;

FIG. 97 illustrates a screen shot showing fields to be manually entered for a patient controlled activation;

FIG. 98 illustrates a screen shot showing numerical entry of values for a patient controlled activation;

FIG. 99 illustrates a screen shot showing alerts and alarms;

FIG. 100 illustrates a screen shot showing selection and/or de-selection of alarms;

FIG. 101 illustrates a screen shot showing selection of sounds for alarms;

FIG. 102 illustrates a screen shot showing pending prime bolus changes not yet downloaded;

FIG. 103 illustrates a screen shot showing pending bridge bolus changes not yet downloaded;

FIG. 104 illustrates a screen shot showing patient controlled activation pending changes not yet downloaded;

FIG. 105 illustrates a screen shot showing an alternative embodiment of pending bridge bolus changes not yet downloaded;

FIG. 106 illustrates a screen shot showing an alternative embodiment of pending prime bolus changes not yet downloaded;

FIG. 107 illustrates a screen shot showing pending periodic changes not yet downloaded;

FIG. 108 illustrates a screen shot showing pending changes not yet downloaded;

FIG. 109 illustrates a screen shot showing a summary of pending changes not yet downloaded;

FIG. 110 illustrates a screen shot showing a warning that an undo will reset values;

FIG. 111 illustrates a screen shot showing a summary of changes to be downloaded;

FIG. 112 illustrates a screen shot showing a table of old values and new values;

FIG. 113 illustrates a screen shot showing an alternative embodiment of a table with old values and new values; and

FIG. 114 illustrates a screen shot showing a report screen.

### DETAILED DESCRIPTION OF THE INVENTION

Implantable medical device 16 can be any of a number of medical devices such as an implantable therapeutic substance delivery device, implantable drug pump, implantable electrical stimulator, cardiac pacemaker, cardioverter or defibrillator, as examples. For purposes of illustration, the present invention will be described mainly with respect to an implantable drug infusion device. However, it should be recognized and understood that the present invention has applicability to other types of implantable medical devices, e.g., implantable electrical stimulators.

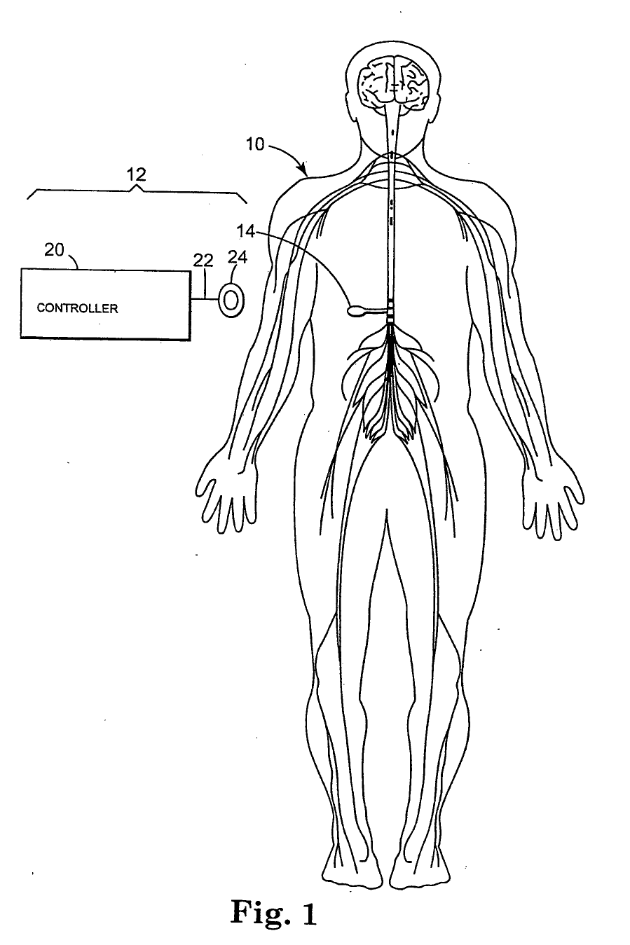

FIG. 1 is a schematic view of drug infusion system 12 of the present invention. Implantable drug infusion device 14 is shown implanted within the body of patient 10. Drug infusion device 14 is programmable through a telemetry link from controller 20, which is coupled via a conductor 22 to a radio frequency antenna 24. Drug infusion device 14 could be, but is not limited to being, a pump for infusing fluid medication into a patient's body. Methods of communicating, using radio frequency telemetry, with implanted treatment devices in order to program such implanted drug infusion devices, are well known in the art.

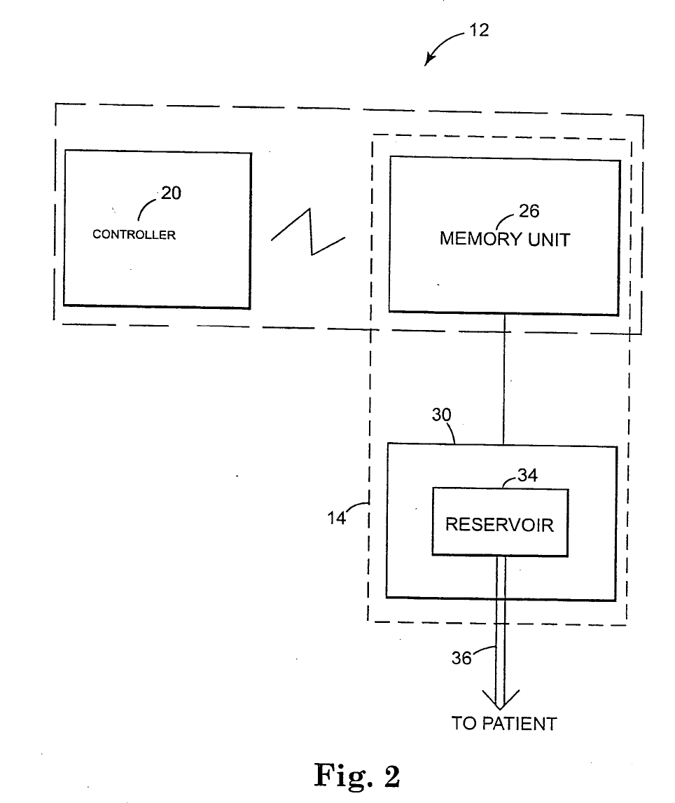

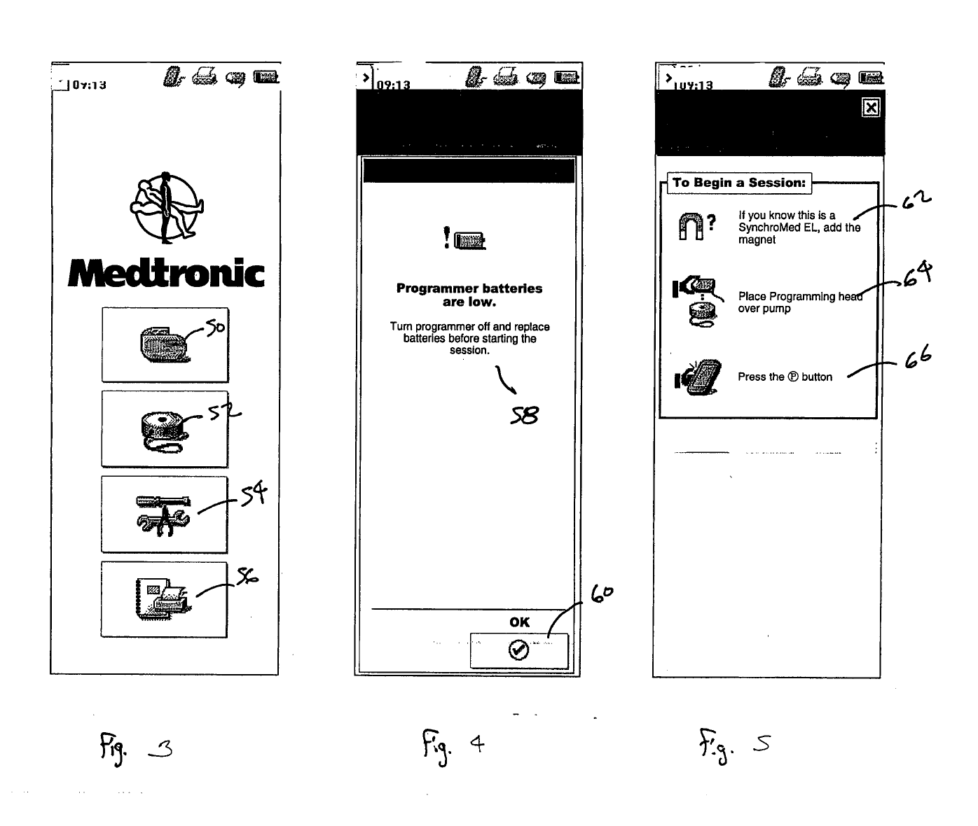

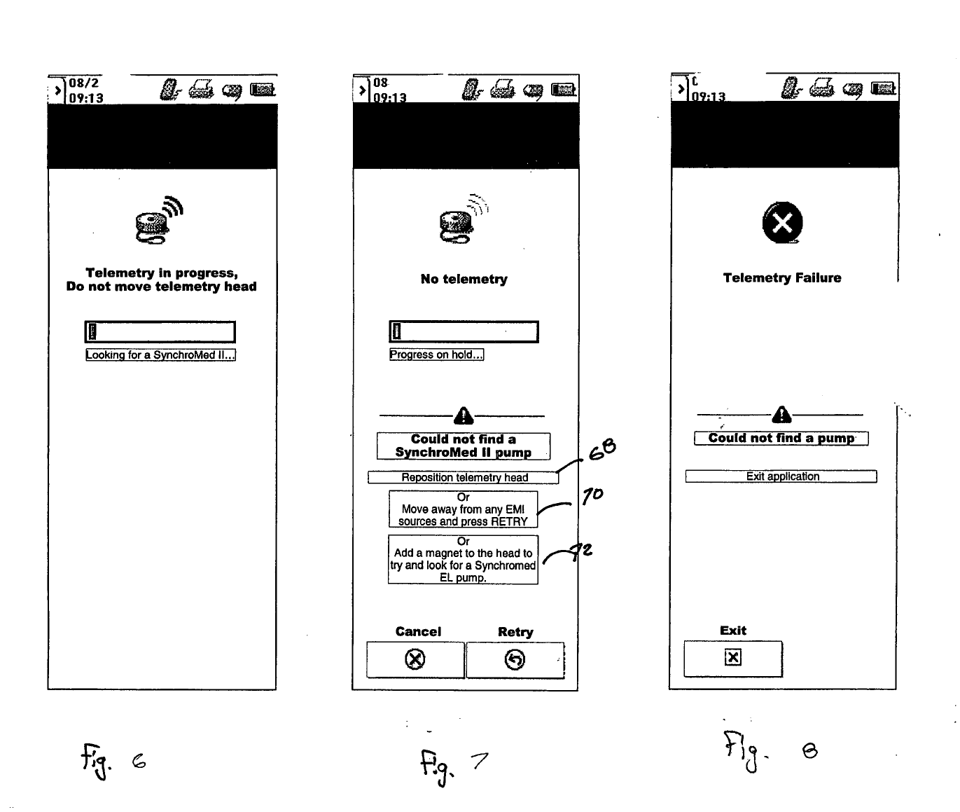

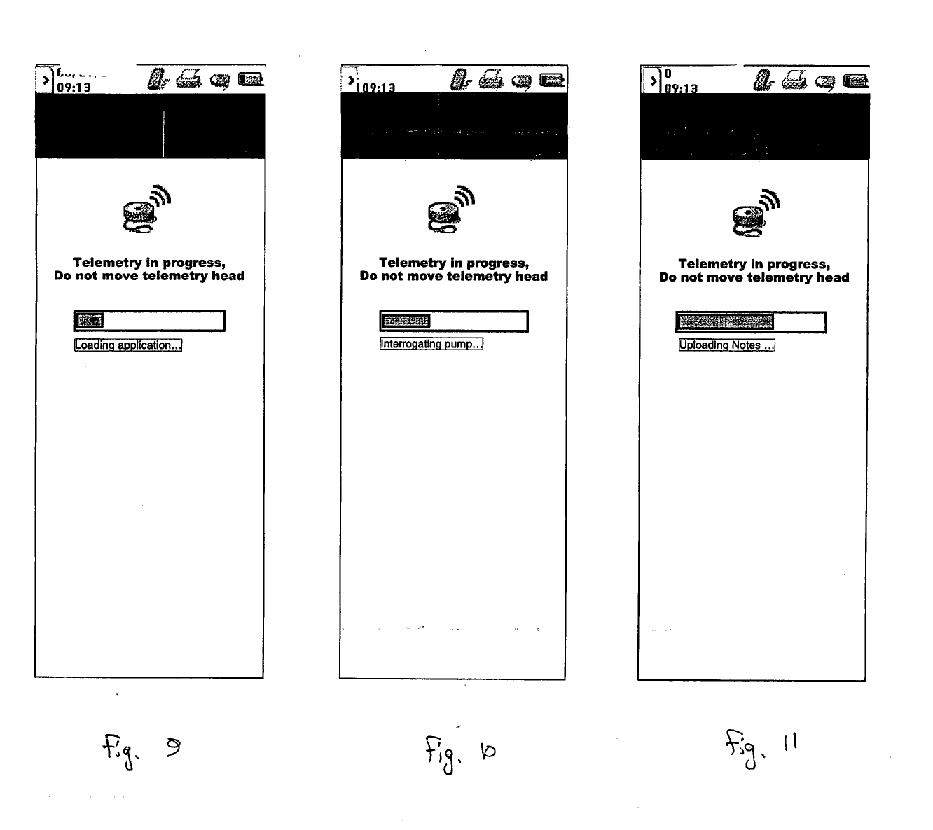

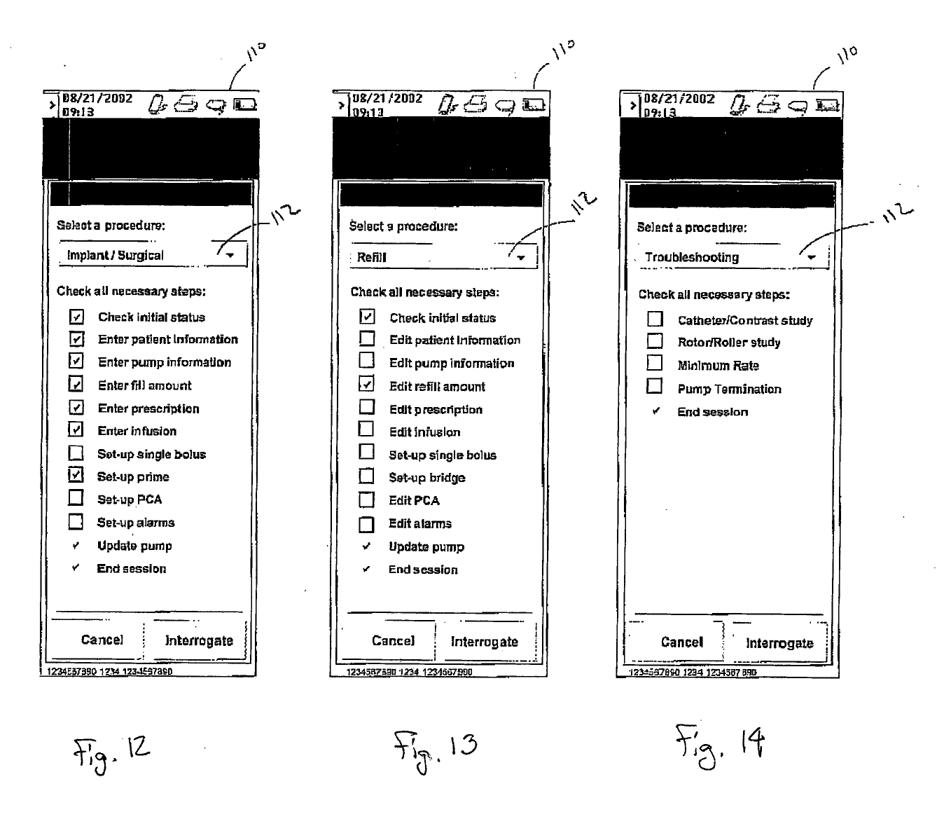

FIG. 2 is a block diagram of drug infusion system 12 having an implantable drug infusion device 14. Drug infusion device 14 consists of an internal memory unit 26 containing memory and registers which provide internal drug delivery instructions to drug delivery module 30. External controller 20 acts as an input-output device for drug infusion system 12 and also provides computational support for memory unit 26. Memory unit 26 and controller 20, operating together, function to control drug delivery module 30 in the delivery of fluid medication to patient 10. In general, drug delivery module 30 is a pump for infusing a fluid medication, including a drug or a combination of drugs, to patient 10. Drug delivery module 30 has a reservoir 34 for holding the fluid medication to be infused and is coupled to patient 10 through catheter tubing 36. Such drug delivery modules 30 are well known in the art.

Memory 26 receives programming information, via telemetry, from controller 20 through conventional means. Programming information, once stored in memory unit 26, provides the dosing regimen to be performed by drug delivery module 30.

Controllers 20 capable of interacting with drug infusion devices are well known in the art. Similarly, techniques for non-invasively communicating between controllers 20 and implanted drug infusion devices, such as by telemetry, are also well known.

Controller 20 typically requires certain inputs of data or information from a medical professional in order to adequately and fully control an implanted medical device. These types of information input can range from patient information, e.g., to keep track of programming regimens among various patients, implantable medical device type and model, and perhaps serial number, capacity or reservoir size, catheter volume, implantation date and implantation location and/or orientation, as well as information related to the programmability functions of the implanted medical device. If the implanted medical device is a drug infusion device, information may need to be input or obtained regarding fluid medication prescription, kinds and amounts or concentrations of fluid medications, amount of fluid medication filled into the reservoir, the infusion program including constant or variable dosage, daily changes, patient administered options such as boluses. Further, information may also be needed regarding a special initial infusion, commonly referred to as a prime bolus, to account for the initial volume of fluid contained in the catheter which may or may not be the same as the fluid medication contained in the reservoir. Still further, upon refilling the implanted drug infusion device with a new supply of fluid medication, information may be needed regarding a special interim infusion, commonly referred to as a bridge bolus, to account for any change in kind or concentration of fluid medication. Alarms may need to be programmed or set or silenced regarding various anomalies that may occur during programming or infusion. And still further, information may need to be supplied or displayed regarding refill procedures, such as the estimated time to refill or estimated time to battery replacement or explanation. These types of items are generally referred to as tasks throughout this description.

As can be seen, there are quite a few variables involving quite a bit of information. The amount and diverse nature of this information can be somewhat bewildering to a medical professional, especially a medical professional who is not intimately familiar with the implanted drug infusion or with controller 20. This may require the medical professional to take more time to be thorough in programming the device and may cause the medical professional to take more time than necessary to accomplish the task.

While the medical professional may not be totally familiar with the particular implanted drug infusion device or with controller 20, the medical professional is usually very familiar with the medical procedures that need to be accomplished. For example, the medical professional will typically understand that a certain medical procedure needs to be accomplished. As an example, the medical professional knows that the drug infusion device has been newly implanted into the patient and needs to be set up and initially programmed. As another example, the medical professional may know that the drug infusion device has just been refilled with a different fluid medication, kind or concentration, and needs to be re-programmed. It can be recognized that each of these medical procedures may require different sets of tasks to be performed in order to accomplish the particular procedure involved. For example, an initial implantation procedure may require data to be input regarding the patient's name and particulars. However, during a refill procedure, information regarding the patient may not need to be reviewed or modified unless a change has occurred, as by a name change associated with marriage, for example. As another example, an initial implantation procedure typically will require the use of a prime bolus but not a bridge bolus. Conversely, a refill procedure may require the use of a bridge bolus but not a prime bolus.

The interfaces associated with controller 20 of some embodiments of the present invention assist in clarifying and streamlining the tasks needed to be performed by the medical professional. A medical professional accomplishing an initial implantation procedure need not be confused with screens involving a bridge bolus. Similarly, a medical professional accomplishing a refill procedure need not be bothered with screens involving a prime bolus.

The following screen shots will illustrate the interface associated with controller 20 in assisting a medical professional in performing the tasks needed in order to accomplish the medical procedure desired.

In FIG. 3, a user may select from one of four different functional areas. Implantable drug infusion device icon 50 may be selected to perform functions related to programmability of implantable drug infusion device 14. Telemetry icon 52 may be selected to perform functions related to the establishment and maintenance of telemetry between controller 20 and implantable drug infusion device 14. Tools icon 54 may be selected to perform utility and maintenance operations. Report icon 56 may be selected to display, view, transmit and/or print, either to file, function (such as facsimile, electronic message, for example) or to a hard copy printer (such as a paper printer or other hard copy output device).

FIG. 4 provides a warning screen shot 58 informing the user that batteries of controller 20, also referred to as a programmer, are low in voltage, capacity, remaining usable life or other battery characteristic. The user may be instructed to turn the controller 20 off and/or to replace or otherwise replenish the battery capacity, e.g., by recharging the batteries in the controller 20. The start up screen depicted in FIG. 4 may be cleared by selected the “OK” icon 60.

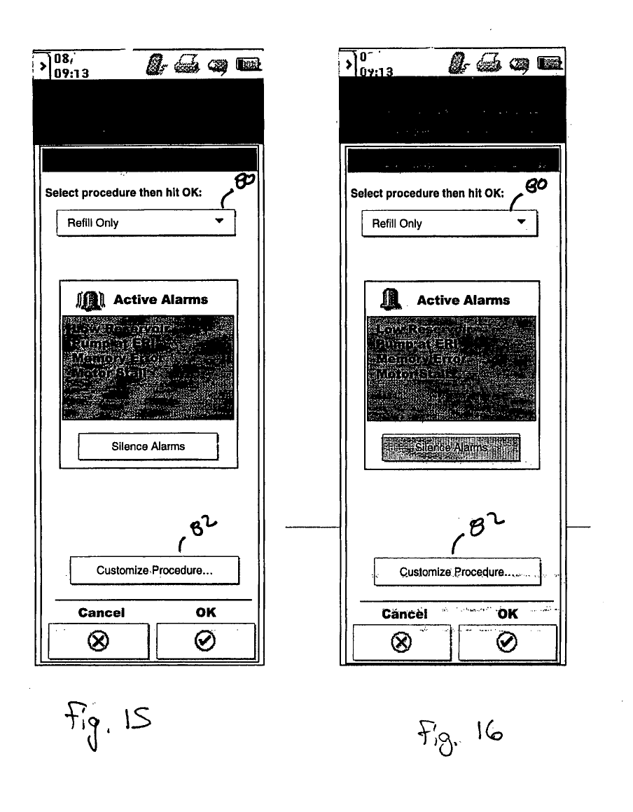

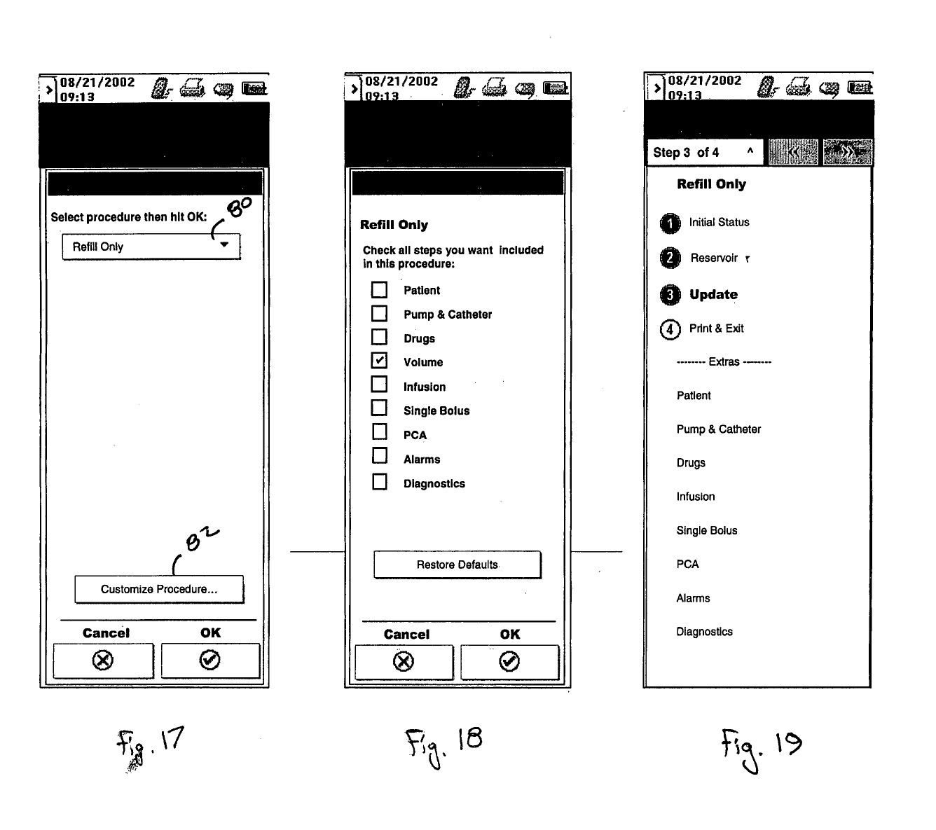

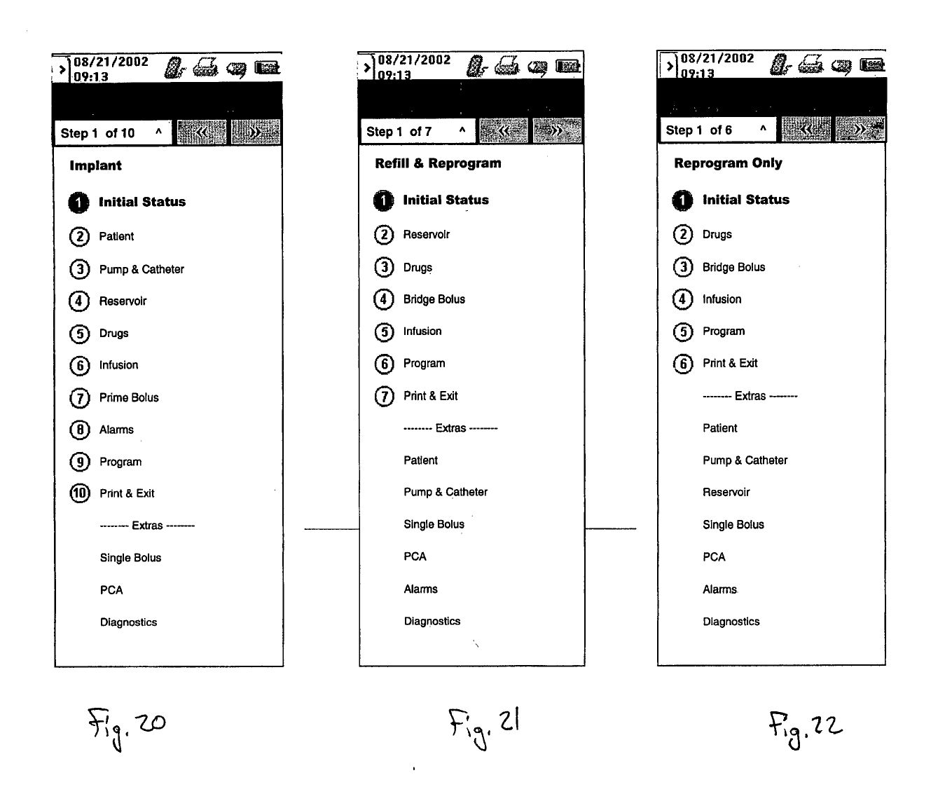

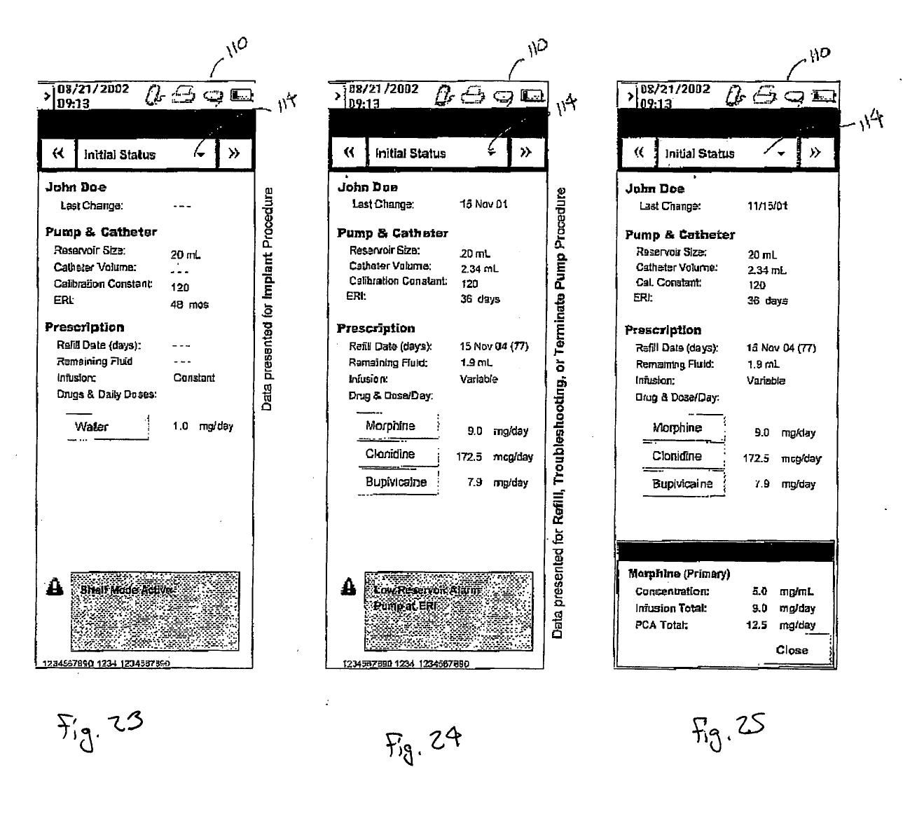

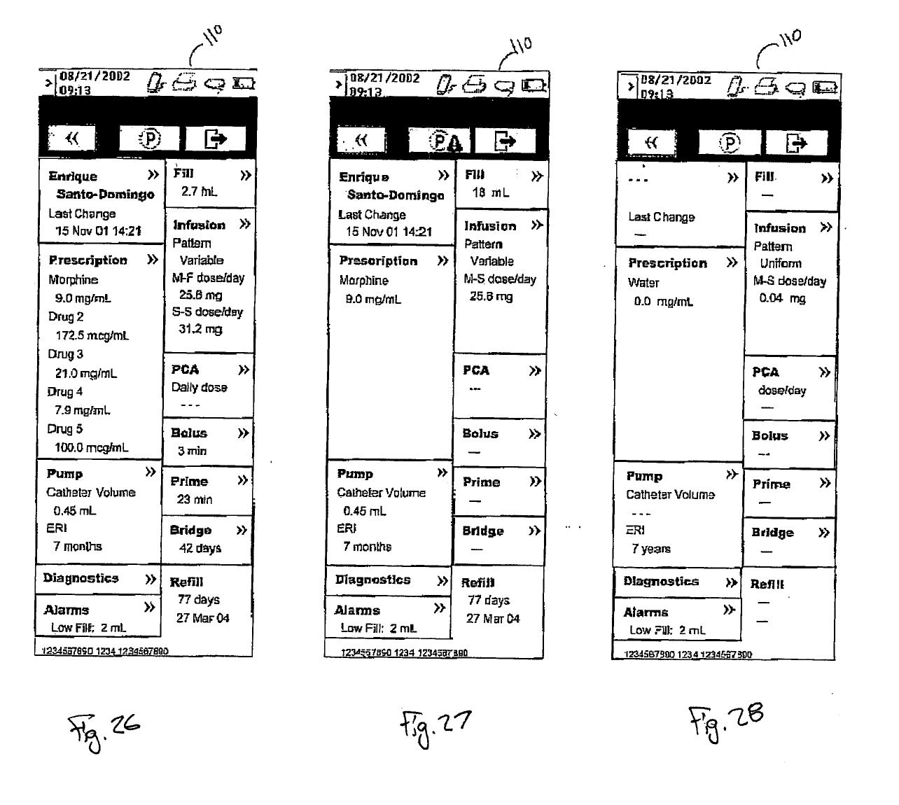

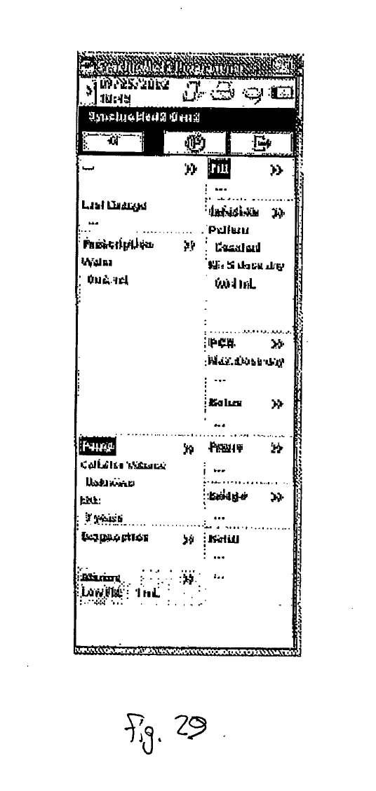

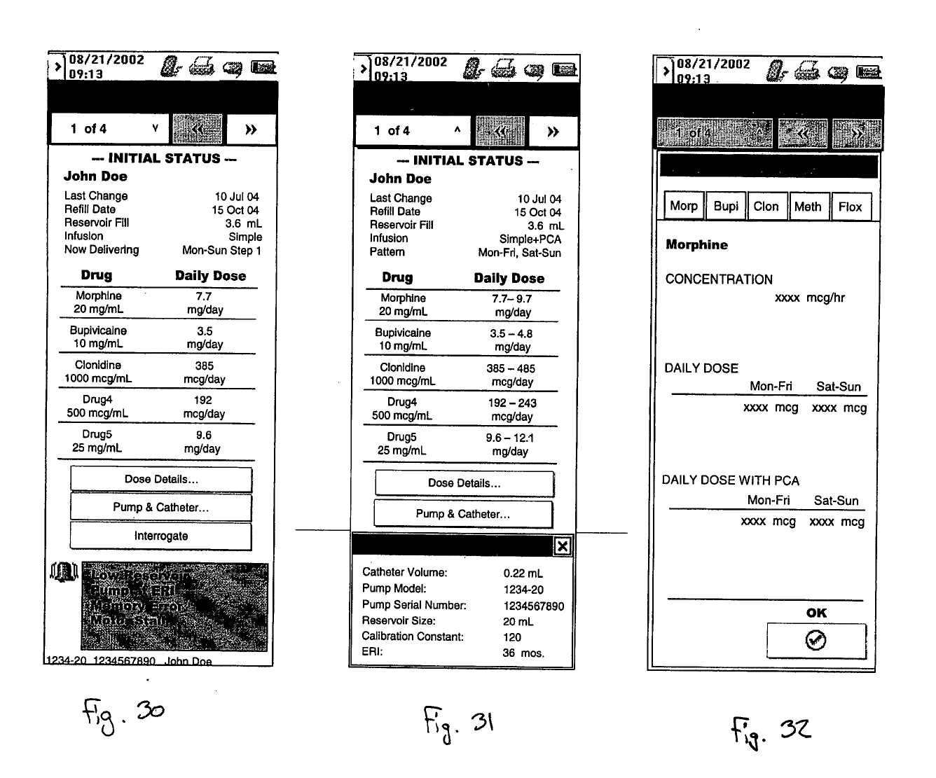

FIG. 5 is an informational screen shot providing instructions for a user to begin a telemetry session with the implantable drug infusion device 14. In step-by-step instructions, the user is led through the process of establishing a telemetry session. In step 62, a user may be instructed to perform a preliminary step, such as utilizing or configuring the controller 20, perhaps dependent upon the type or model of controller 20 and/or implantable drug infusion device 14. In step 64, the user is instructed to place the programming head of the controller 14, e.g., the antenna, over the location in the body 12 where implantable drug infusion pump 14 is implanted. In step 66, the user is instructed to press a button to initiate programming of implantable drug infusion device 14.

In FIG. 6, Controller 14 then displays the screen shot illustrated in FIG. 6 informing the user that the controller 14 is searching for an implantable drug infusion device 14 with which to establish telemetry. The user is informed that telemetry is in process and is instructed not to move the telemetry head (antenna). If telemetry could not be established with implantable drug infusion device 14, a screen shot as illustrated in FIG. 7 may be displayed communicating an inability to establish telemetry. The screen shot illustrated in FIG. 7 may also provide guidance to the user to correct the problem with establishing telemetry, such as by repositioning the telemetry head, antenna, 68, moving away from sources of electromagnetic interference 70 or to reconfigure 72 the controller 14, such as by adding an accessory magnet.

Once telemetry is established, the information screen shots illustrated in FIG. 9, FIG. 10, and FIG. 11 are displayed indicating that telemetry between controller 20 and implantable drug infusion device 14 is in progress. The user may be instructed not to the telemetry head, antenna. In FIG. 9, telemetry is in progress and an application is loading. In FIG. 10, telemetry is in progress and implantable drug infusion device 14 is being interrogated. In FIG. 9, telemetry is in progress and notes are being uploaded from implantable drug infusion pump 14 to controller 20.

FIG. 12, FIG. 13 and FIG. 14 illustrate screen shots 110 of controller 20 implementing a task based flow interface. Instead of listing all tasks which can be performed on controller 20, the interfaces illustrated in FIG. 12, FIG. 13, and FIG. 14 only list tasks that are pertinent to a procedure that has been selected. In this embodiment, controller 20 presents an interface in which the medical professional selects a procedure to be performed. In this example, three separate and distinct procedures are possible, namely an “implant/surgical” procedure, a “refill” procedure and a “troubleshooting” procedure. The medical professional selects a procedure with the drop-down selection box 112. This selection is accomplished using a standard drop-down box 112 in which the down arrow is selected with a cursor, mouse or other pointer, a list of the three possible procedures are shown and the medical professional selects one of the procedures with the pointer.

FIG. 12 illustrates a screen shot 110 with an “implant/surgical” procedure having been selected. The “implant/surgical” procedure would typically be selected by the medical professional following an initial implantation of a medical device or a surgical revision of an implanted medical device. A list of possible tasks are shown below with only those tasks that are expected to be pertinent to the “implant/surgical” procedure having pre-filled check boxes. In this case, “check initial status”, “enter prescription information”, “enter pump information”, “enter fill amount”, “enter prescription”, “enter infusion” and “set-up prime” tasks are pre-checked. The medical professional has the option of either checking additional tasks or unchecking tasks already checked but the medical professional is initially presented with only the tasks deemed necessary to complete the procedure selected. Two additional tasks are pre-checked, namely “update pump” and “end session”, but cannot be unchecked by the medical professional because these tasks are not optional.

FIG. 13 illustrates a screen shot 110 with a “refill” procedure having been selected by the medical professional. The “refill” procedure would typically be selected upon refilling (or re-programming) an already implanted medical device. Since much of the information required would have already been entered, only two tasks, namely “check initial status” and “edit refill amount”, are pre-checked in addition to the required “update pump” and “end session” tasks. Normally, patient information will not need to be updated and the pump will not have changed so those tasks are not pre-checked. The medical professional, of course, has the option to check other tasks if desirable.

FIG. 14 illustrates a screen shot 110 with a “troubleshooting” procedure having been selected. In this screen shot 110 a different list of tasks are presented with no task pre-checked except for the mandatory “end session” task. The particular troubleshooting task or tasks can then easily be checked by the medical professional as needed.

Note that in each instance, only tasks which are possible for the procedure selected are presented and only those tasks expected to be performed are pre-checked. This simplifies the programming tasks for the medical professional by streamlining what is expected.

Once the medical professional has confirmed those tasks that are to be performed, the medical professional selects the “interrogate” button to continue the programming process. Alternatively, the medical professional could instead select the “cancel” button to be returned to the procedure selection screen.

FIG. 15 and FIG. 16 illustrate an alternative embodiment of a task based flow interface. In FIG. 15, the procedure to be performed by the user can be selected from drop down list 80. The screen shot of FIG. 15 is illustrated with alarms activated as indicated by the “ringing bell.” The screen shot of FIG. 16 is illustrated with alarms silenced as indicated by the “static bell.”

FIG. 17 is similar to the screen shots of FIGS. 15 and 16 with the procedure to be performed selected by way of the drop down list 80. However in FIG. 17, no alarms are displayed.

In either of FIGS. 15, 16 or 17, the procedure to be performed may be customized by selecting the “customize” icon 82.

Once the “Refill Only” procedure is selected in either of FIGS. 15, 16 or 17, the screen shot illustrated in FIG. 18 may be presented to the user. The screen shot of FIG. 18 contains a list of possible procedural steps which may be included in the performance of the overall procedure selected, in the illustrated case shown, a “Refill Only” procedure. Only steps which are related to the selected procedure, i.e., “Refill Only,” are contained in the list presented in FIG. 18. If a different overall procedure had been selected in the introductory screen of either of FIGS. 15, 16 or 17, then different procedural steps may be listed in the screen shot represented by FIG. 18. The procedural steps desired to be performed by the user are individually checked before the “OK” icon is selected. Alternatively, individual procedural steps may be selected on a group basis.

FIG. 19 illustrates a screen shot of the status of “Refill Only” procedure. In the screen shot illustrated, steps 1 (initial status), 2 (reservoir) and 3 (update) have been completed. Step 4 (print & exit) remains to be performed. Also illustrated in the screen shot of FIG. 19 are possible extra steps which could also be performed during the “Refill Only” procedure such as creating or modifying information regarding the patient, the pump & catheter, drugs contained in the implantable drug infusion device 14, single bolus, patient controlled activation, alarms and diagnostics.

FIGS. 20, 21 and 22 illustrate the procedural steps as in FIG. 19 for different procedures. FIG. 20 illustrates the procedural steps included within the “Implant” procedure. FIG. 21 illustrates the procedural steps included within the “Refill & Reprogram” procedure. FIG. 22 illustrates the procedural steps included within the “Reprogram Only” procedure.

FIG. 23, FIG. 24 and FIG. 25 illustrate screen shots 110 showing initial status screens of one embodiment reached following selection of the “interrogate” button. FIG. 23 is associated with the “implant/surgical” procedure. FIG. 24 is associated with the “refill” procedure and FIG. 25 is associated with the “troubleshooting” procedure.

The tasks associated with each procedure are chronologically organized. That is, the first screen/task presented following the “interrogate” request is the first task that chronologically needs to be performed. This is identified by the task drop-down 114. In this case, it is the “initial status” task.

On each side of the task drop-down 114 are forward and back buttons. The forward button will take the medical professional to the next task, which has been checked, to be logically chronologically performed. Hitting the forward button again takes the medical professional to the next chronological task. The back button does the opposite, taking the medical professional to the previous chronological task. Presenting the tasks in this order allows the medical professional to merely keep progressing to the next task with the forward button without having to think about which task in the next chronological task.

In an embodiment, the task drop-down box 114 is a true drop-down box. When the drop-down is selected, the box shows all of the possible tasks that may be performed on controller 20, or at least more tasks than were pre-checked or individually checked at the initial procedure screen. This allows the medical professional the flexibility of navigating to any task at any time even though certain tasks have been pre-selected or selected for a particular procedure.

In a preferred embodiment, drop-down task box 114 will show tasks that have been previously visited, whether by forward/back navigation or drop-down box selection, with a distinct visual representation, e.g., with a grayed background. This visual confirmation can aid the medical professional in remembering which tasks have already been visited and which have not.

FIG. 26, FIG. 27 and FIG. 28 illustrate screen shots 110 showing an alternative interface for controller 20. All three screen shots illustrate an “all in one” interface in which all applicable tasks are shown in summary form on one screen shot 110. The medical professional may then see at a glance a summary of the status of all applicable tasks and may navigate to any desired task directly simply by selecting, for example by tapping, the particular task. FIG. 26, FIG. 27 and FIG. 28 represent slightly different statuses of implanted medical device. FIG. 26 illustrates the status of an implanted infusion drug device containing multiple drugs with multiple boluses set. FIG. 27 illustrates the status of an implanted infusion drug device containing a single drug and no boluses set. FIG. 28 illustrates the status of a drug infusion device either before implantation or following implantation but before initial parameters have been input.

Task areas that are recommended to be visited by the medical professional for the medical procedure being performed may be visually distinctly identified on the main screen. For example, the “pump” task area and the “fill” task area may be presented in reverse text.

When the medical professional navigates to a task area by selecting, e.g., tapping, on the arrows associated with a particular task area, controller 20 then presents an individualized screen applicable to that task area. These task areas are described below. When the medical professional has completed a task area, or otherwise desires to return to the main screens illustrated in FIGS. 26, 27 or 28, the medical professional need only select the “exit door” featured on every task area screen to return to the main screens. After visiting a task area and returning to the main screen, the task areas that have been visiting may be distinctly identified in the main screen, such as by a grayed background, as illustrated in FIG. 29.

FIG. 30 illustrates an initial status screen shot providing information about the name of the patient, information concerning the date of last change of to the programming of the implantable drug infusion device 14, the last refill date, the quantity of the reservoir fill, the type of infusion and the pattern. Further, the initial status screen shot illustrated in FIG. 30 provides a summary of information concerning the drugs contained in the reservoir of the implantable drug infusion device 14 and the amount of the daily dose of each drug. Dose details, pump and catheter information and information concerning the implantable drug infusion device 14 itself may be obtained by selecting or interrogating the lower icons on the screen. FIG. 30 illustrates exemplary warnings related to low reservoir, pump error, memory error and motor stall.

FIG. 31 illustrates a similar screen shot as that illustrated in FIG. 30 but with information displayed as a result of interrogation of the details concerning implantable drug infusion device 14.

FIG. 32 illustrates the screen shot resulting from clicking on or otherwise selecting the drug “morphine” in the initial status screen of FIG. 30. The specific concentration of the drug contained in the reservoir may be displayed along with the base daily dose and daily dose with all available patient controlled boluses.

FIG. 33, FIG. 34 and FIG. 35 illustrate screen shots of the patient task area. Navigation to this area occurs by selecting, e.g., tapping, the arrows associated in the patient task area of one of the initial status screens. In FIG. 33, detailed patient information may be provided. In FIG. 34, symbol 84 may be provided next to information that has been modified in the current programming session or since a selected or otherwise predetermined time or date. In FIG. 35, a note about the patient may be provided.

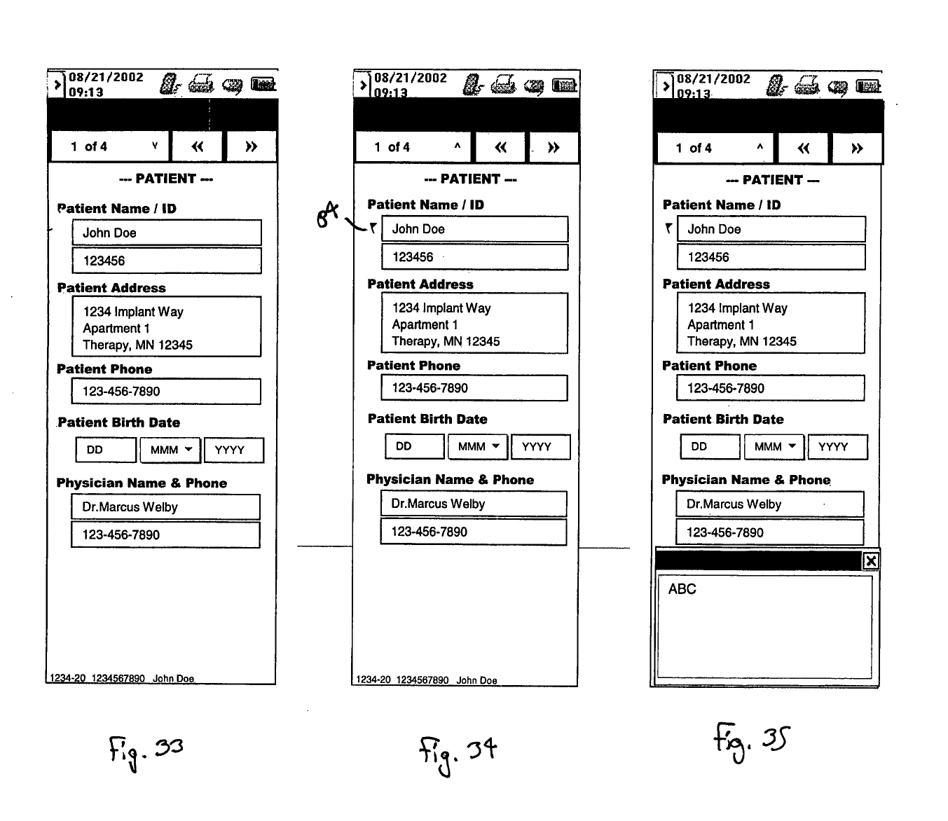

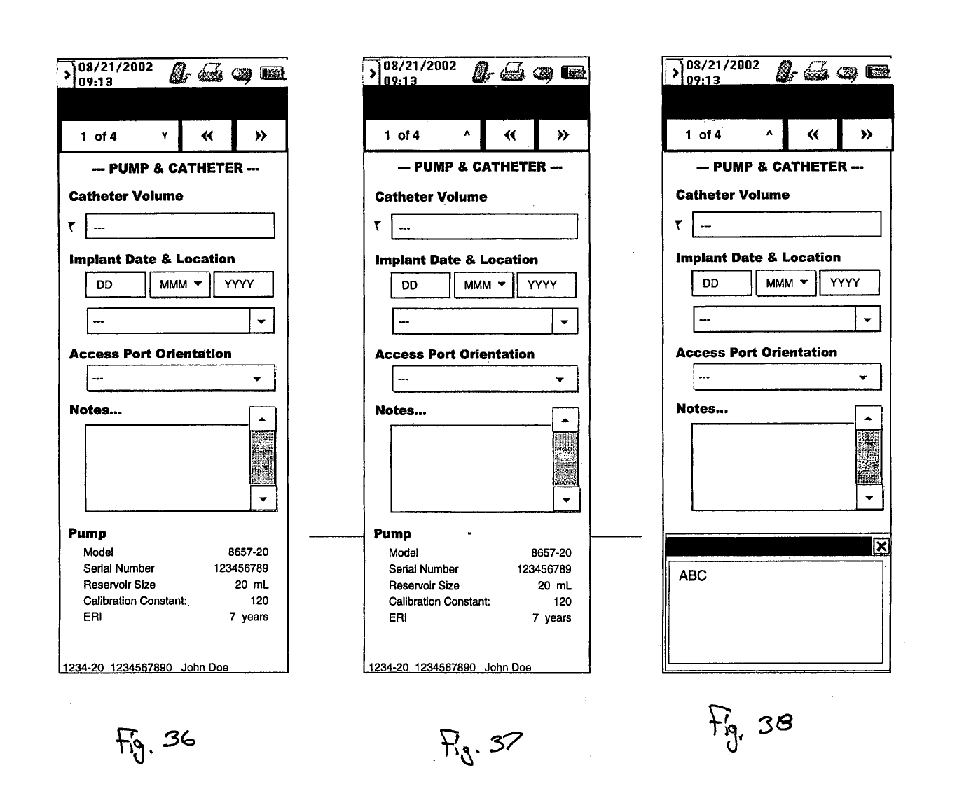

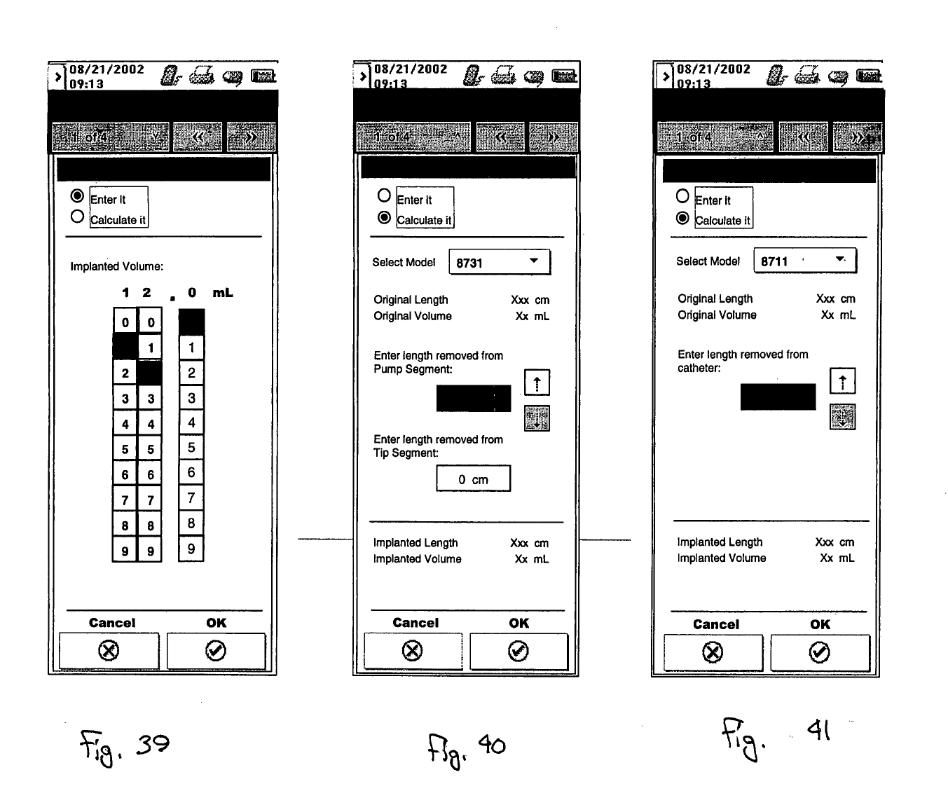

FIGS. 36 through 41 illustrate screen shots from the pump and catheter task area. These screens may be accessed by selecting, e.g., tapping, the arrows associated with pump and catheter task area of one of the initial status screens. In FIGS. 36 through 38, detailed pump information may be provided included model and serial number, implant location, implant orientation, catheter information and notes for future reference. In some cases, at least part of this information will already be known, having been obtained directly from the implanted medical device, e.g., pump model, serial number, reservoir size, calibration constant and ERI. In FIGS. 39 through 41 information concerning the length of the catheter may be inputted by making the selection and/or by tapping the numbers provided in the screen. In FIG. 37, further information about the catheter, necessary for proper implementation of prime and bridge boluses, may be provided. In FIG. 38, information concerning the length removed from the pump segment may be provided.

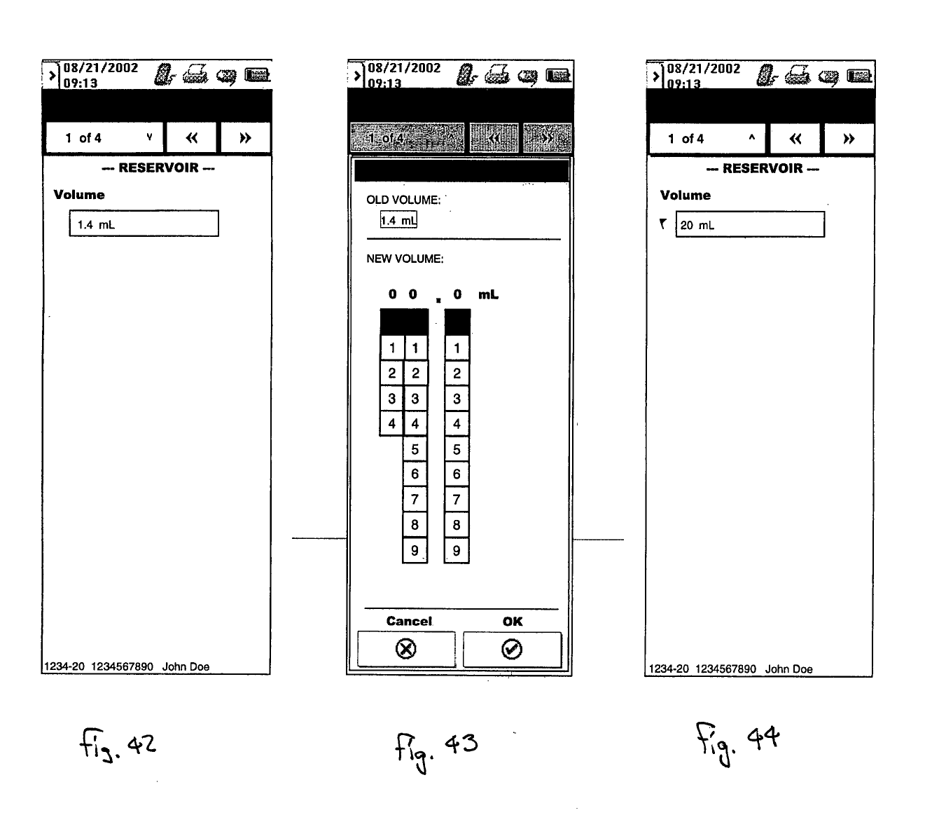

FIGS. 42 through 44 provide information concerning the amount of drug contained in the reservoir of implantable drug infusion device 14. In FIG. 42, the existing volume is displayed. As a change is made in the volume, the screen shot of FIG. 43 is displayed showing the old volume while allowing a new volume to be input. Finally, FIG. 44 displays the new volume in the reservoir following input of FIG. 43. The screen shot of FIG. 44 displays the modified icon next to the modified volume amount.

FIGS. 45 through 56 provide information about and a mechanism to input information concerning the drugs contained in the reservoir of implantable drug infusion device 14. FIG. 45 illustrates that the reservoir simply contains water. FIG. 46 shows a screen with the water of FIG. 45 having been replaced by a single drug, morphine, with a concentration of 25 mg/mL. In FIG. 47, the reservoir contains three drugs, namely morphine, at 25 mg/mL, as in FIG. 46, and with Bupivacaine, at 25 mg/mL, and clonidine, at 250 mg/mL.

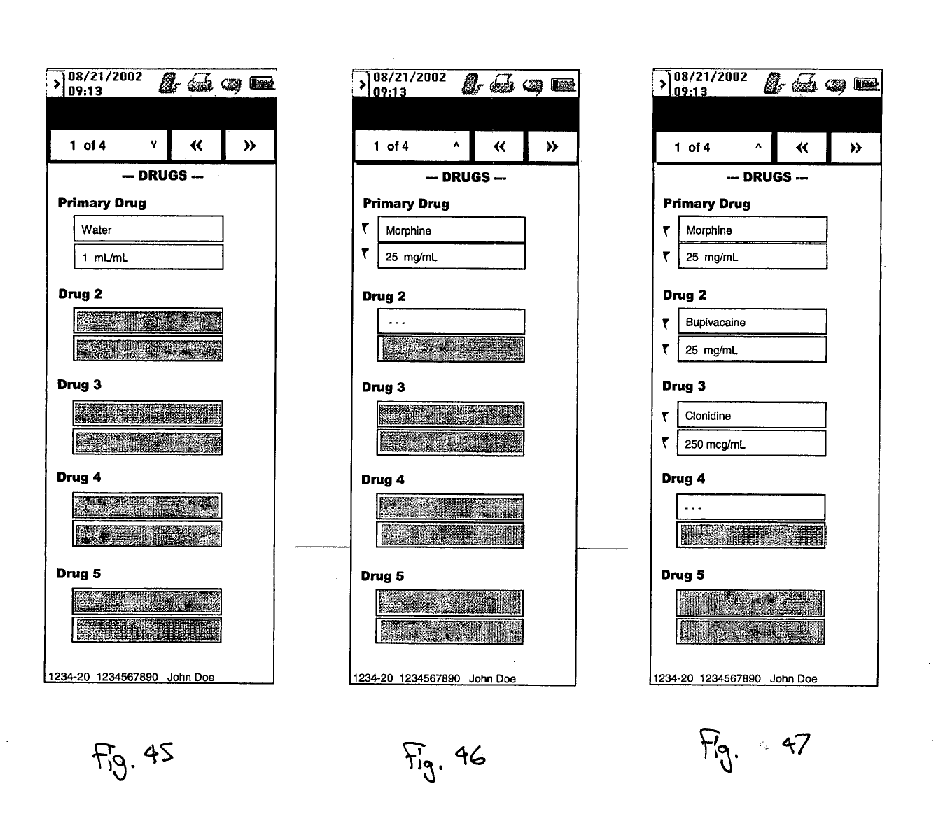

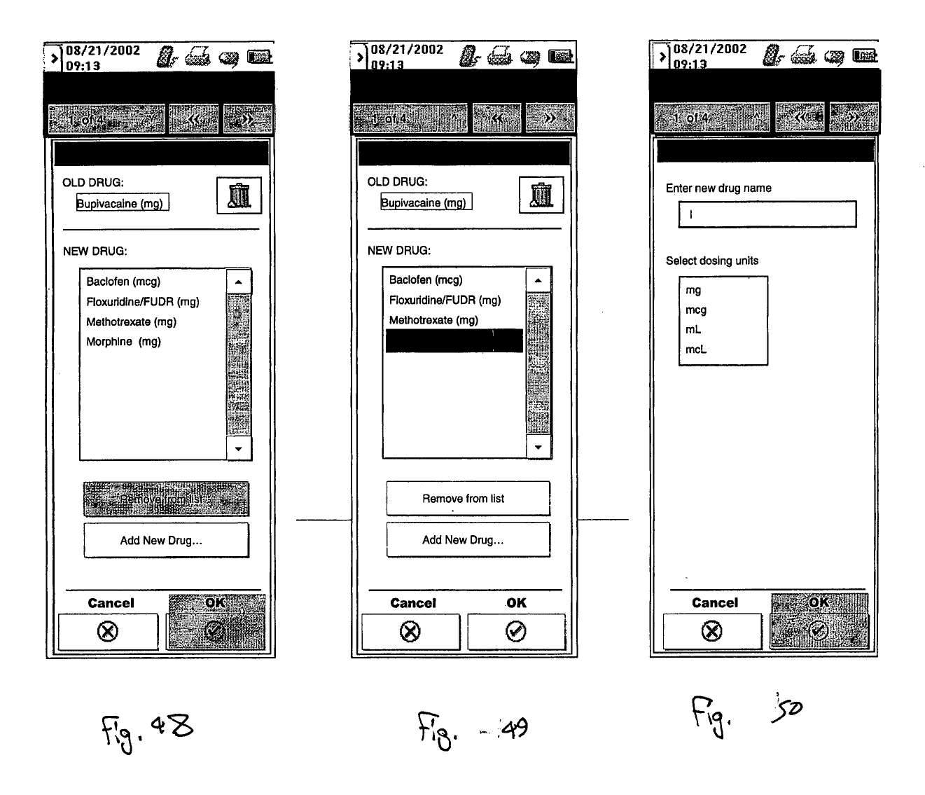

FIG. 48 illustrates the process for replacing a drug entry with a different drug. The old drug is identified in the top and a new drug can be selected from the lower list. New drugs can be added to the list or, alternatively, existing drugs in the list may be removed. In FIG. 49, the drug morphine has been selected and is indicated as having been selected by appearing in reverse type. FIGS. 50 and 51 illustrate the process of entering a new drug name for the drug list including the units of concentration.

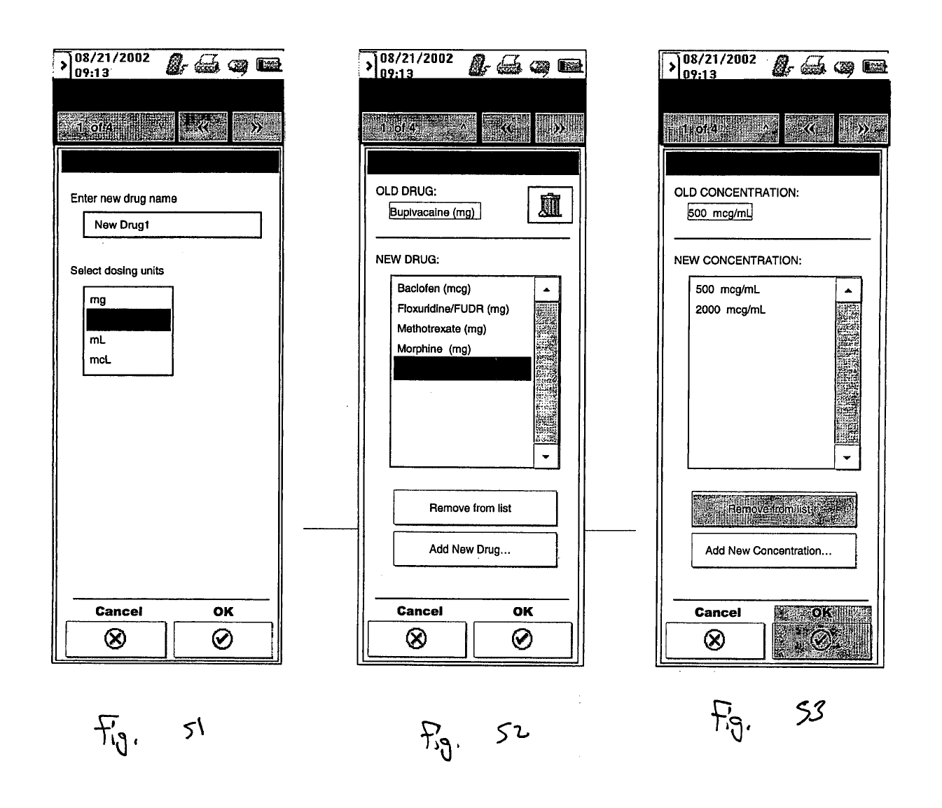

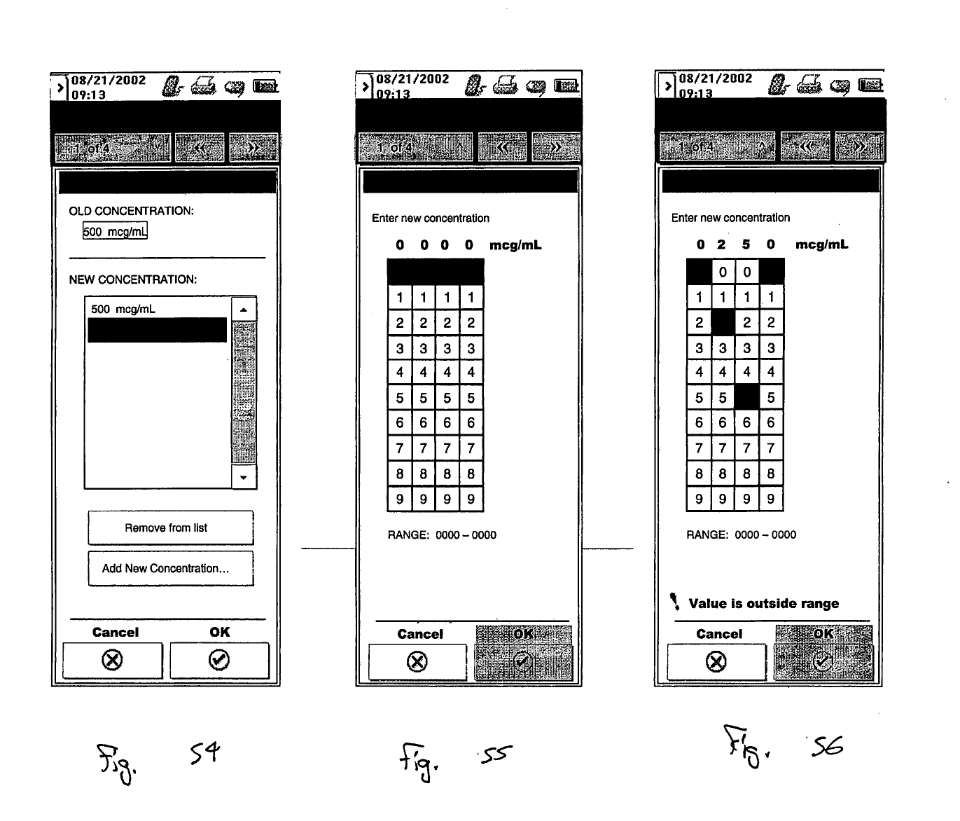

FIGS. 51 through 56 illustrate further the process of entering drug information including selecting a dosing unit (concentration) (FIG. 51), selecting the newly listed drug (FIG. 52), selecting a concentration for the newly selected drug (FIGS. 53 and 54) and inputting a new concentrate amount from which to select (FIGS. 55 and 56).

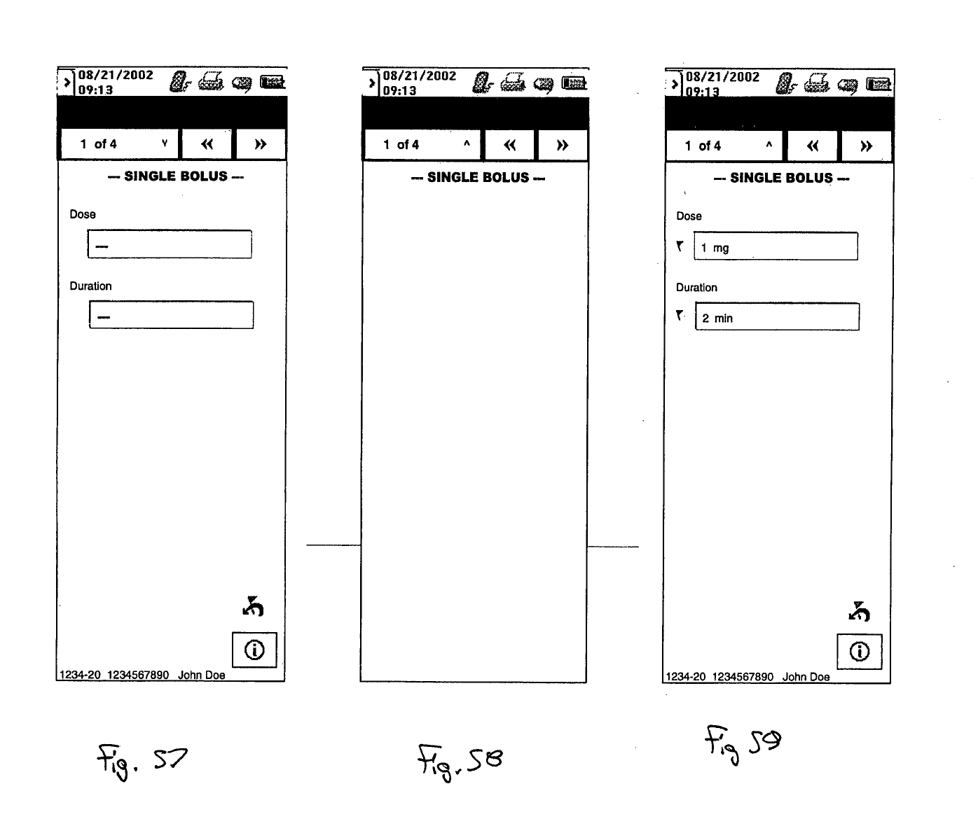

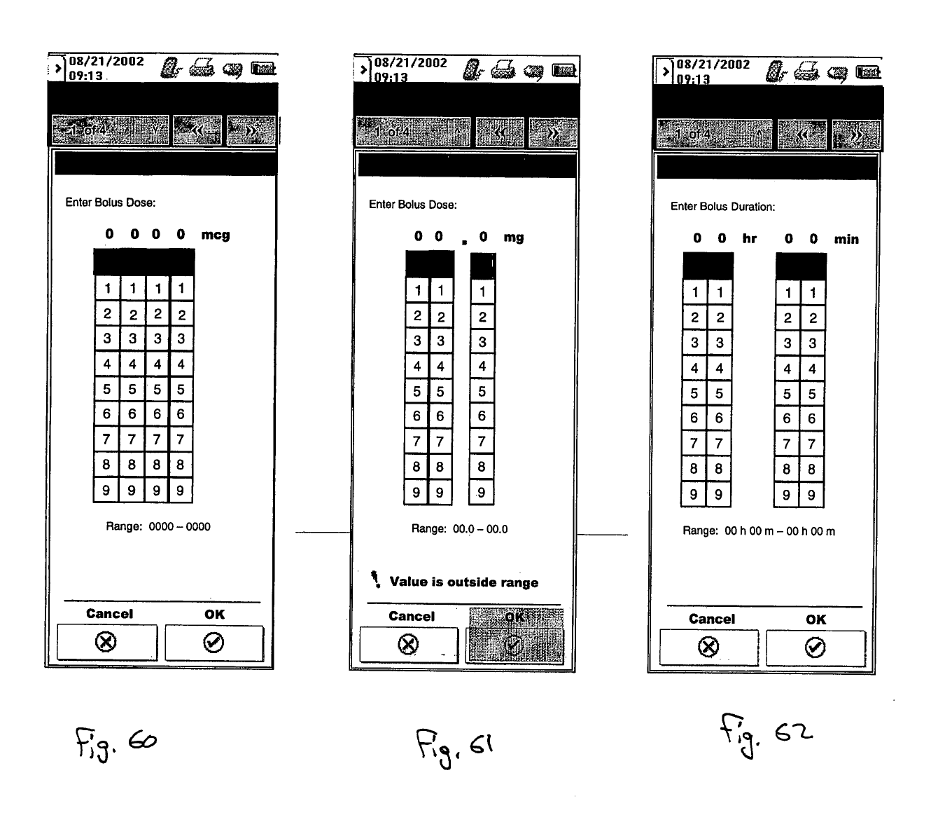

FIGS. 57 through 63 illustrate the process of programming implantable drug infusion device 14 with a single bolus. In FIG. 57, the existing programmed bolus is displayed. In this example, a bolus has not been programmed, hence, the dose and duration of the bolus is blank. Alternatively, the lack of a programmed bolus could be displayed by essentially no detail information following the heading of “bolus” as in FIG. 58. FIG. 59 illustrates the display of a bolus having a dose of 1 milligram with a duration of 2 minutes. FIG. 60 illustrates a screen shot for inputting a bolus dose amount with a warning in FIG. 61 that the value of the bolus attempted to be input is outside of an acceptable range. The bolus duration is input in the screen shot of FIG. 62.

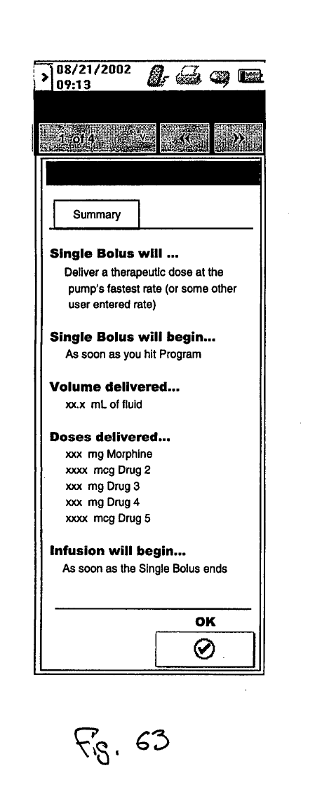

FIG. 63 illustrates a screen providing in conversational language a summary of the bolus parameters existing for implantable drug infusion device 14. The conversational language specifies that the single bolus will deliver a therapeutic dose at the pump's fastest rate (or some other entered rate). The single bolus will begin immediately as soon as the program button/icon is selected. The volume to be delivered is specified. The doses of each drug contained in the reservoir of implantable drug infusion device 14 is calculated and displayed in the screen. Regular infusion will begin as soon as the bolus terminates.

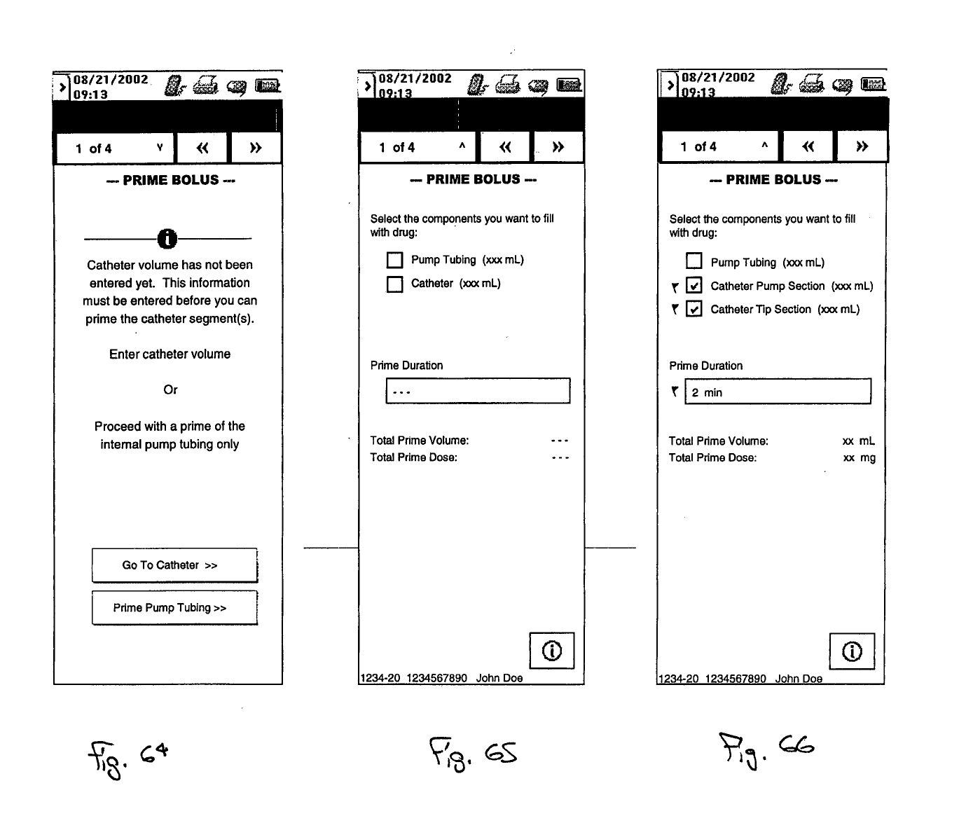

FIGS. 64 through 72 illustrate screen shots associated with programming for a prime bolus, i.e., an initial bolus to be delivered to take into consideration the volume of the catheter recognizing that the catheter must fill with therapeutic solution from the implantable drug infusion device 14 before regular infusion may commence. FIG. 64 reminds the user that the catheter volume has not yet been entered. The option is provided to enter the catheter volume or to proceed with a prime bolus utilizing only the volume of internal pump tubing. In FIG. 65 the components for the prime bolus are selected, i.e., either pump tubing, catheter volume, or both. A prime duration is specified. In FIG. 66, two portions, pump section and tip section, of the catheter volume are selected and a prime duration of 2 minutes is specified.

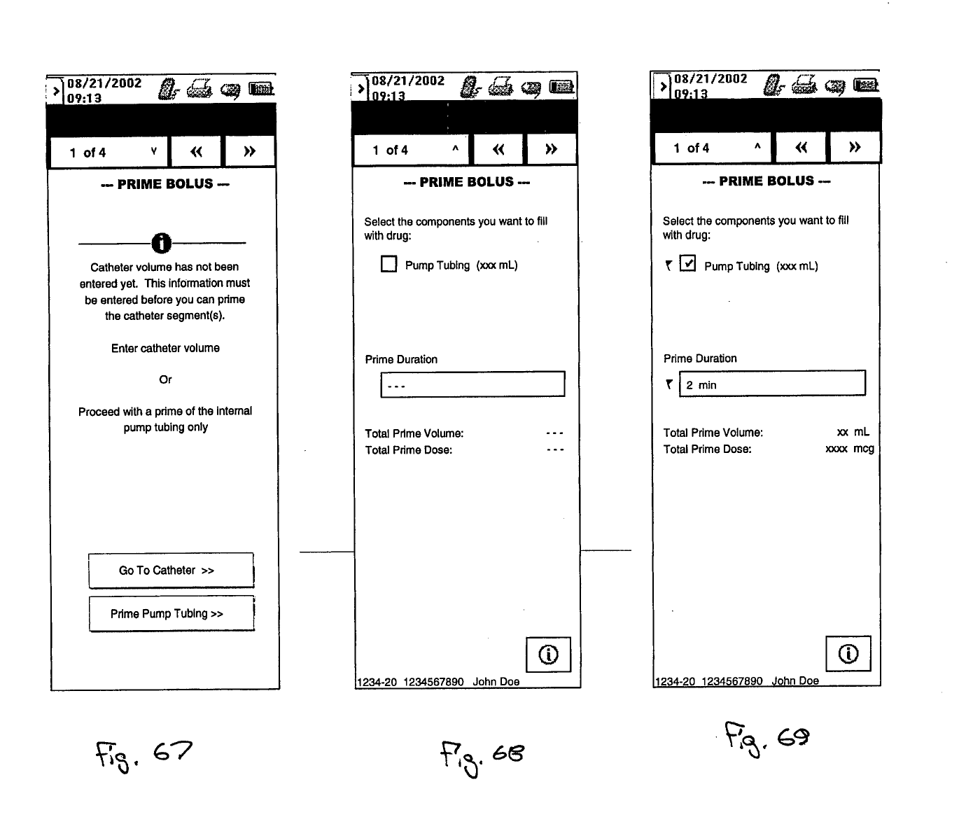

FIGS. 67 through 69 illustrate screen shots for programming a prime bolus similar to that programmed in FIGS. 64 through 66 but instead programming a pump tubing prime bolus only instead of a catheter prime bolus.

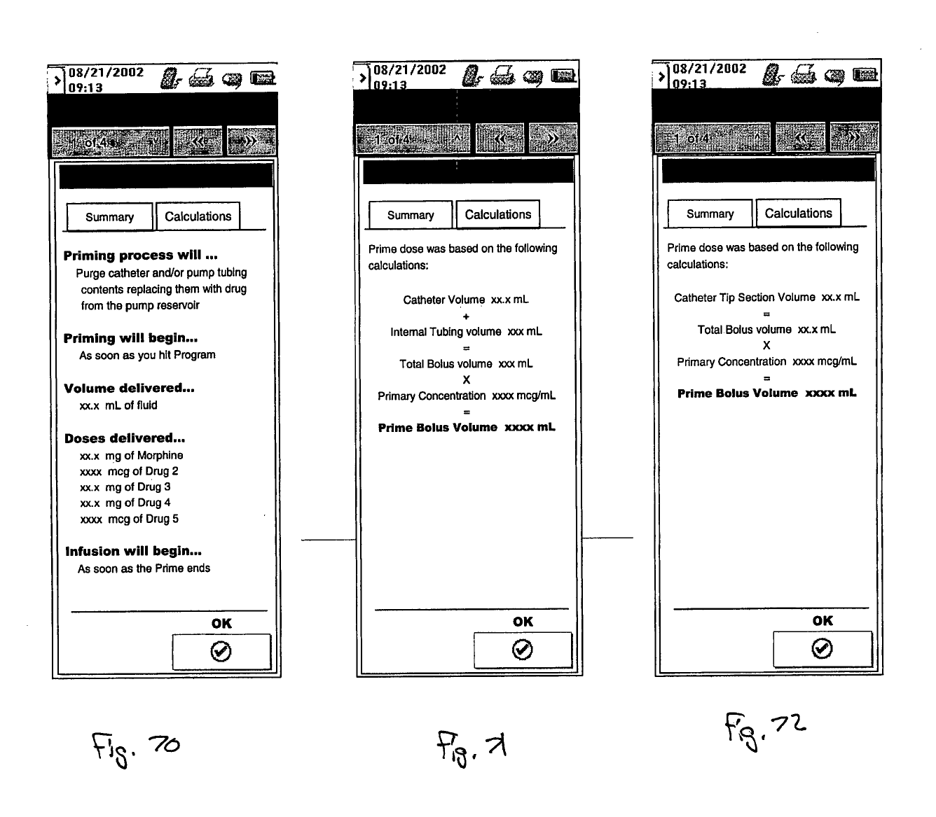

FIG. 70 illustrates a screen shot providing a summary of the prime bolus having been programmed in conversational language. The prime bolus is explained using short sentences, such as “Priming process will purge catheter and/or pump tubing contents replacing them with drug from the pump reservoir” explaining the purpose of the prime bolus. Similarly, information is provided about when the prime bolus will begin, the volume that will be delivered, the doses of each drug delivered and the when base infusion will commence. FIGS. 71 and 72 illustrate alternative embodiments of the contents of the “calculations” tab from the screen shot of FIG. 70 displaying the calculations made by controller 20 in order to obtain the prime bolus volume. The display of the internal calculation allows the user, such as a medical practitioner, to check the appropriateness of the calculation.

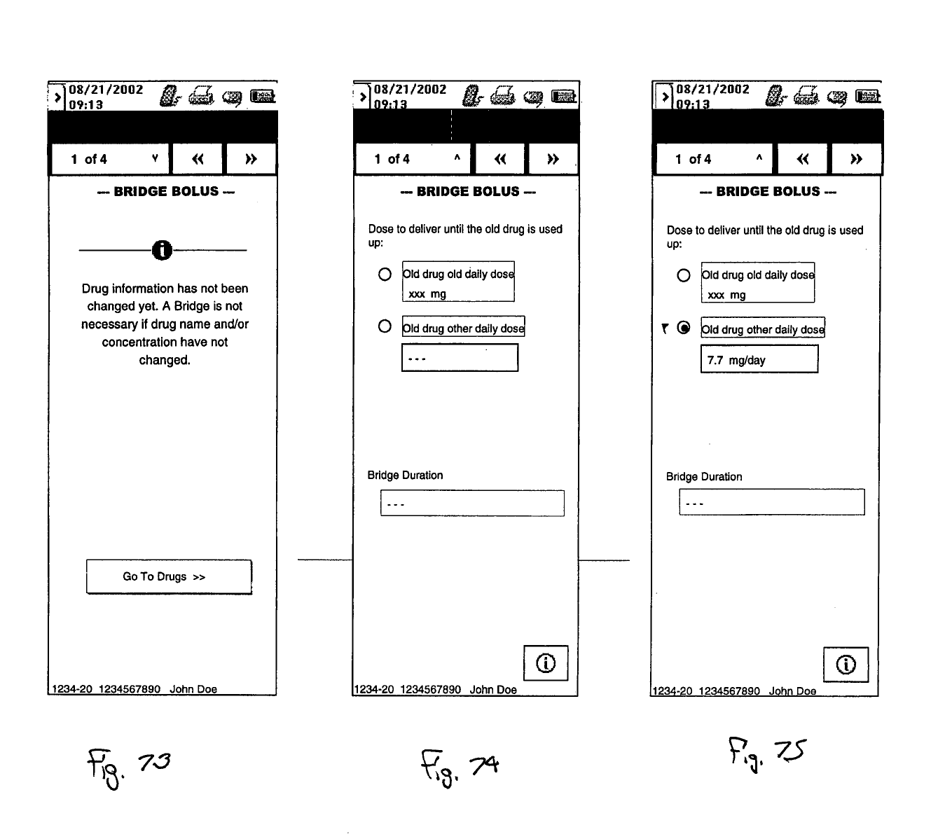

FIGS. 73 through 77 illustrate the screen shots associated with the programming of a bridge bolus. A bridge bolus may be used to take care of infusion during the period when one drug or drugs is being replaced by another drug or drugs. The medical practitioner should ensure that minimum and maximum doses of both the old and new drugs are taken into consideration. In FIG. 73, the user is warned that drug information has not yet been changed and invites the user to go to the steps to change the drug information. FIGS. 74 and 75 illustrate screen shots allowing for the input of the daily dose of the old drug and any other daily dose of the old drug. The daily dose of the new drug is contained in the drug information section of the controller 20 interface. In this case, an other daily dose of 7.7 mg/day has been specified.

FIG. 76 illustrates a screen shot providing a summary of the bridge bolus having been programming in conversational language. The bridge bolus is explained using short sentences, such as “Bridge process will pump at a temporary rate until the old and/or concentration is used up” explaining the purpose of the bridge bolus. Similarly, information is provided about when the bridge bolus will begin, the volume that will be delivered, the doses of each drug delivered and the when base infusion will commence.

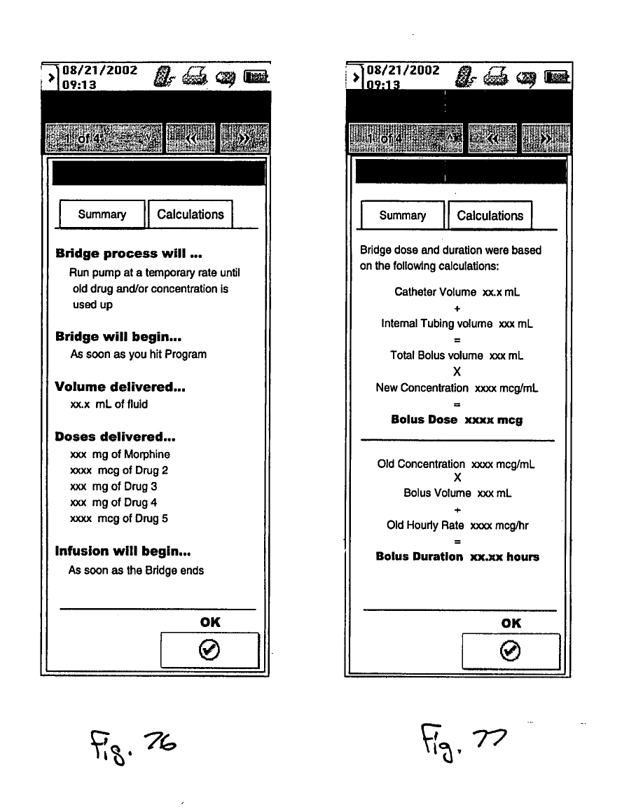

FIG. 77 illustrates the contents of the “calculations” tab from the screen shot of FIG. 76 displaying the calculations made by controller 20 in order to obtain the bridge bolus volume. The display of the internal calculation allows the user, such as a medical practitioner, to check the appropriateness of the calculation.

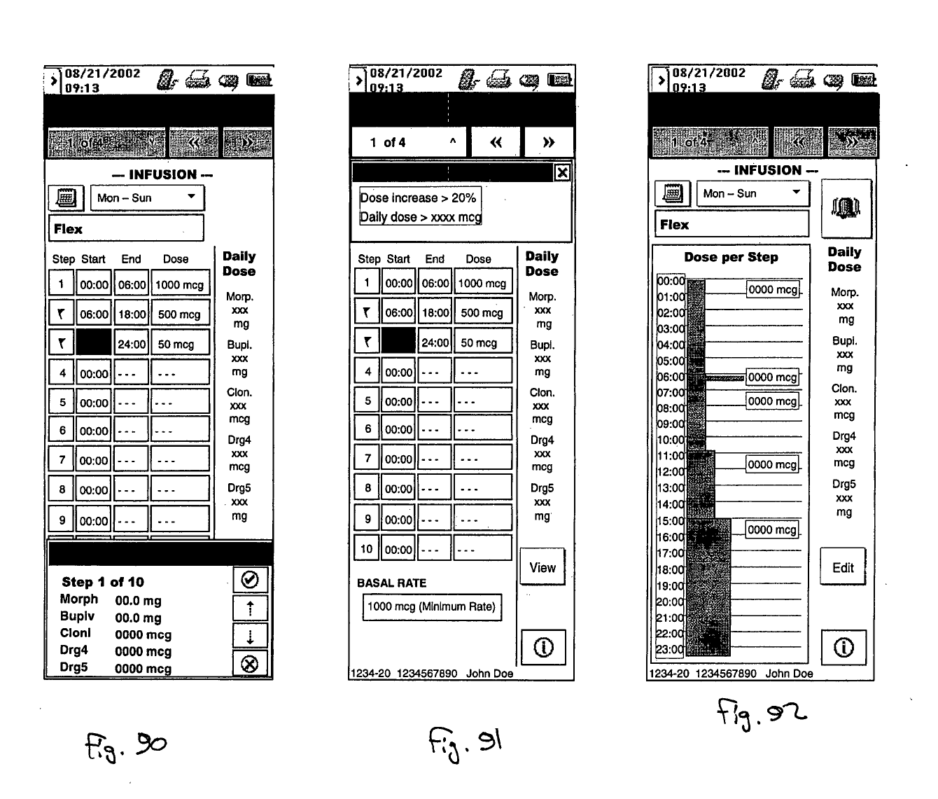

FIGS. 78 through 95 illustrate screen shots associated with programming the drug infusion program. In FIG. 78, the type of drug infusion program is selected. In the example provided, the drug infusion program may be a simple program, periodic program, day-night program, flexible program, a titration program or a minimum rate program. A short explanation of the meaning of each type of program is provided.

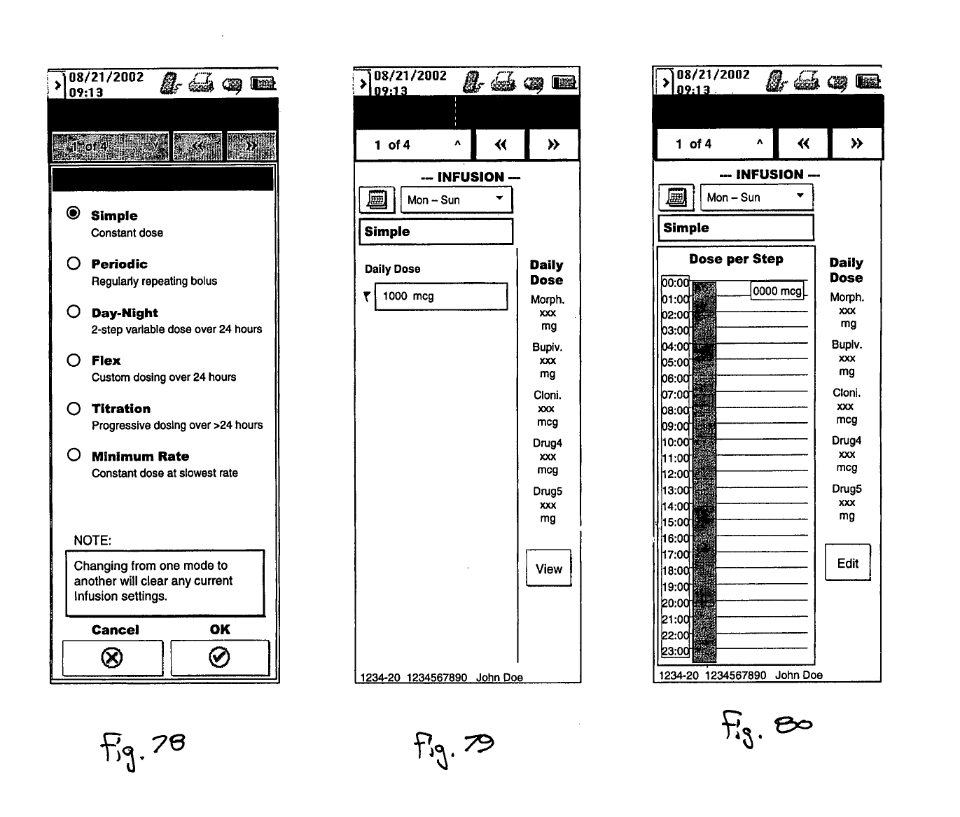

FIGS. 79 and 80 illustrate screen shots associated with programming a simple drug infusion program. A daily dose may be manually input, as in FIG. 79, or a graphical interface may be provided in which the user may graphically view the dose and modify the dose by clicking on the dose bar on the graphical display and dragging the dose to a different dosage, either higher or lower, as in FIG. 80.

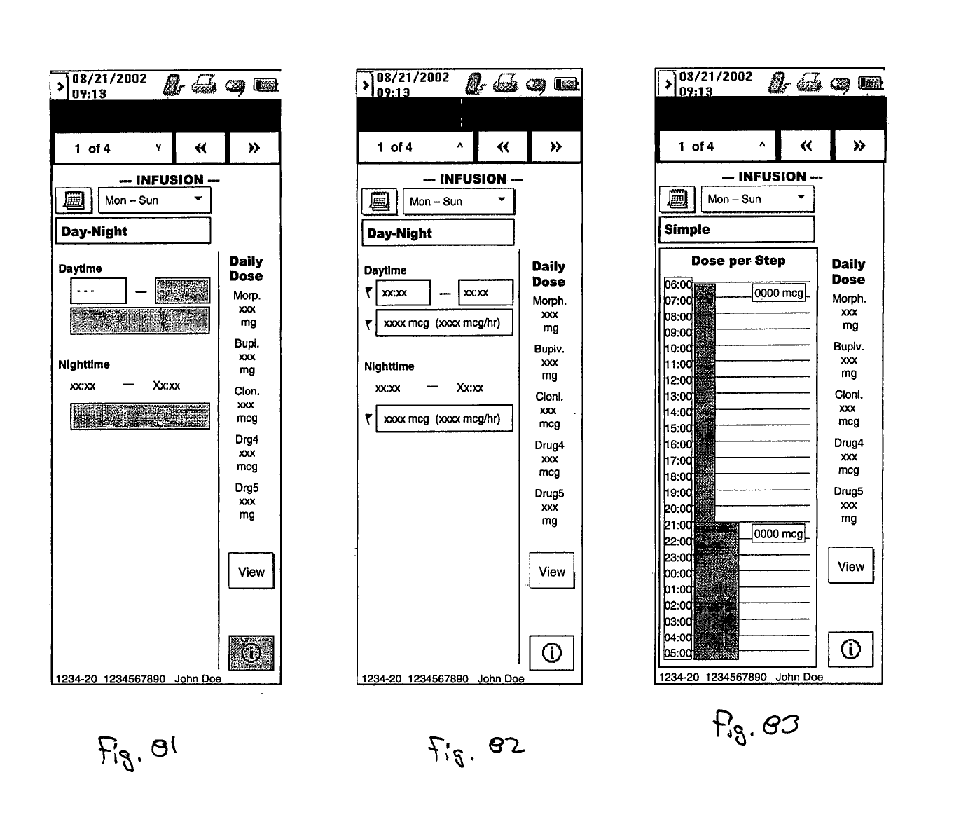

FIGS. 81 through 83 illustrate screen shots associated with programming a day-night drug infusion program. The day-night drug infusion program is selected in FIG. 81. As with the simple drug infusion program, the programmed dosages may be input, as in FIG. 82, or a graphical interface may be provided in which the user may graphically view the dose and modify the dose by clicking on the dose bar on the graphical display and dragging the dose to a different dosage, either higher or lower, as in FIG. 83. The difference is that the day-night drug infusion program contains two components, a day component and a night component. Note that the user may individually drag either the day or night segment to increase or decrease the dosage or the change the time of day that the change-over from day to night and/or night to occur.

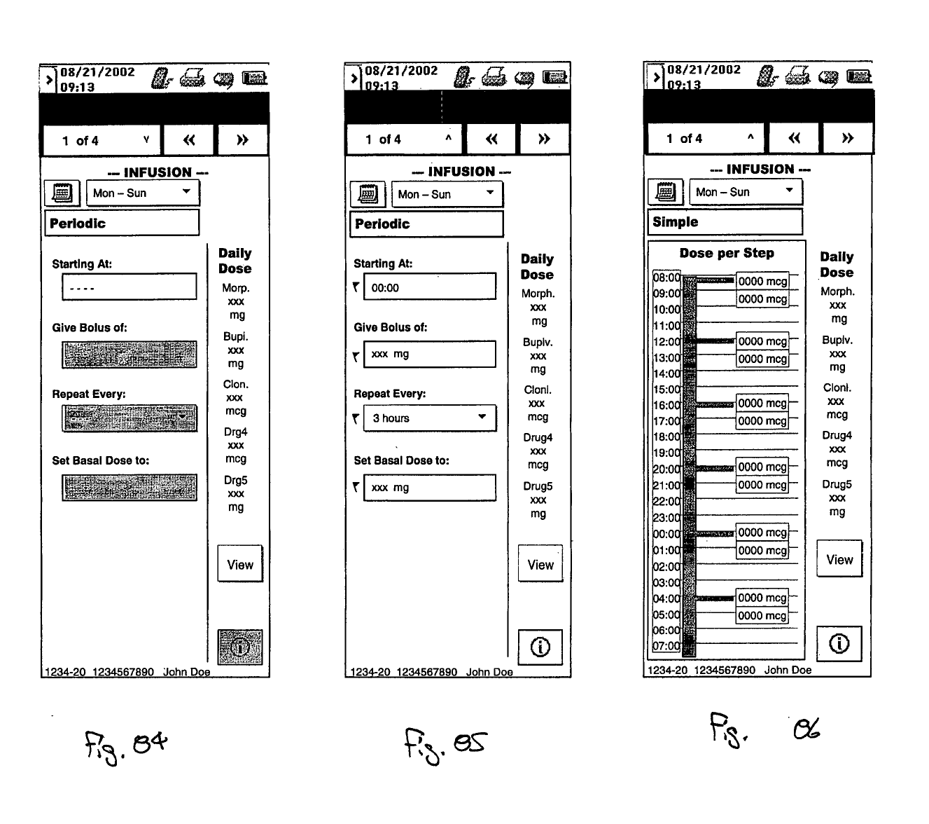

FIGS. 84 through 86 illustrate screen shots associated with programming a periodic drug infusion program. The periodic drug infusion program is selected in FIG. 84. As with the day-night drug infusion program, the programmed dosages may be input, as in FIG. 85, or a graphical interface may be provided in which the user may graphically view the dose and modify the dose by clicking on the dose bar on the graphical display and dragging the dose to a different dosage, either higher or lower, as in FIG. 86. The difference is that the periodic drug infusion program may contain many more components, rather than just a day component and a night component. Note that the user may individually drag any or all of individual segments to increase or decrease the dosage or the change the time of day that each segment occurs.

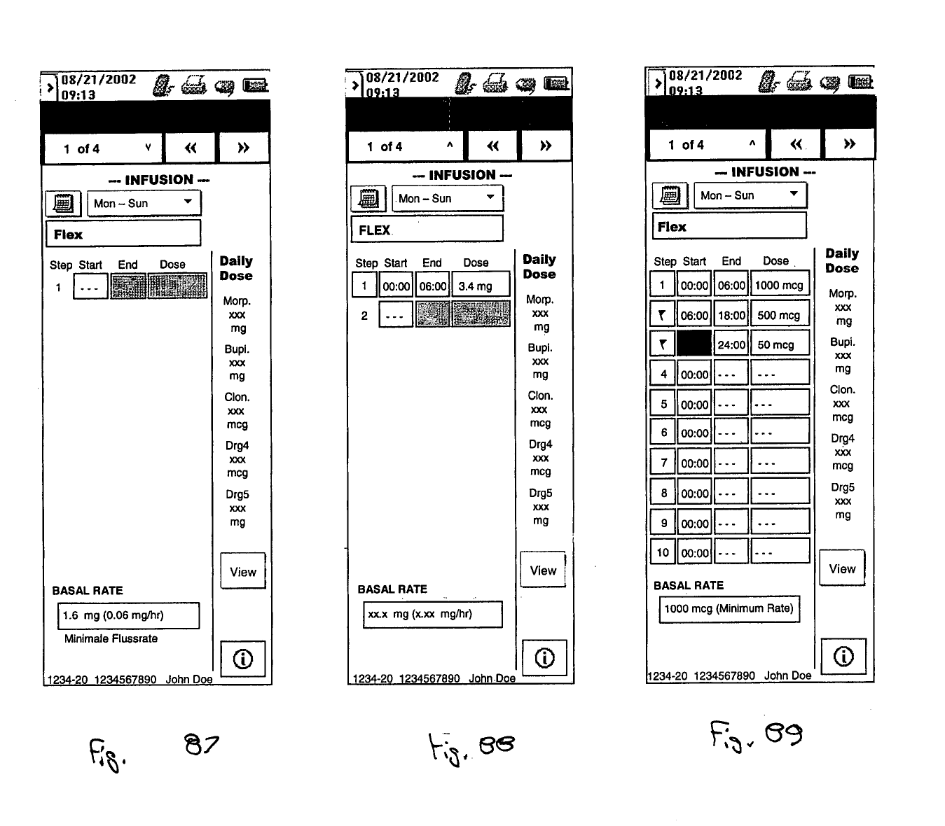

FIGS. 87 through 95 illustrate screen shots associated with programming a flexible drug infusion program. The periodic drug infusion program is selected in FIG. 87. As with the periodic drug infusion program, the programmed dosages for each step of the flexible drug infusion program may be input manually, as in FIGS. 88 through 91, or a graphical interface may be provided in which the user may graphically view the dose and modify the dose by clicking on the dose bar on the graphical display and dragging the dose to a different dosage, either higher or lower, as in FIG. 92. Note that the user may individually drag any or all of individual segments to increase or decrease the dosage or the change the time of day that each segment occurs.

The graphical representation shows the hours of the day in the vertical axis and the dose per hour in the horizontal axis. The exact amount programmed for each portion of the graphical representation is revealed by pausing the cursor over the particular portion of the graph and a pop-up appears with the numeric information. The daily dose for each medication is shown in the right hand margin to provide the medical professional with up-to-date information of the total dosing amounts as the infusion is changed. The graphical representation shown illustrates a continuous dose of less than 1 milligram per hour, perhaps about ¾ milligrams per hour, with two single boluses implemented at about the 0700 hour and the 1500 hour, respectively. These single boluses bring the total dose delivered during each bolus to around 10 milligrams per hour.

The dosage amount may be modified by the medical professional by clicking on the graphical representation (the dose bar) and dragging the dose bar either to right (increase the dosage) or to the left (decrease the dosage). In either case, the amount of the dose per hour and the daily dose immediately reflect the new position of the graphical dose bar.

If a bolus is desired, the medical professional may click in the open area of the graphical representation at a time where the bolus is desired to begin and drag the cursor down to the time where the bolus is desired to end. As the cursor is released, a new graphical segment is created which itself may be dragged left or right to obtain the desired amount of bolus.

In either case, the dosage rate may be changed, or the start or stop times of boluses may be changed simply by clicking and dragging the cursor on the graphical representation. This interface not only provides the medical professional with a bird's eye view of the daily infusion program but also allows the medical professional to modify the infusion program while maintaining that bird's eye view. Individual screens showing start and stop times and manual entry of dosage amounts are not required.

Of course, the medical professional has the option of entering a dosage amount or a bolus amount through individual start and stop times and manual entry of amount.

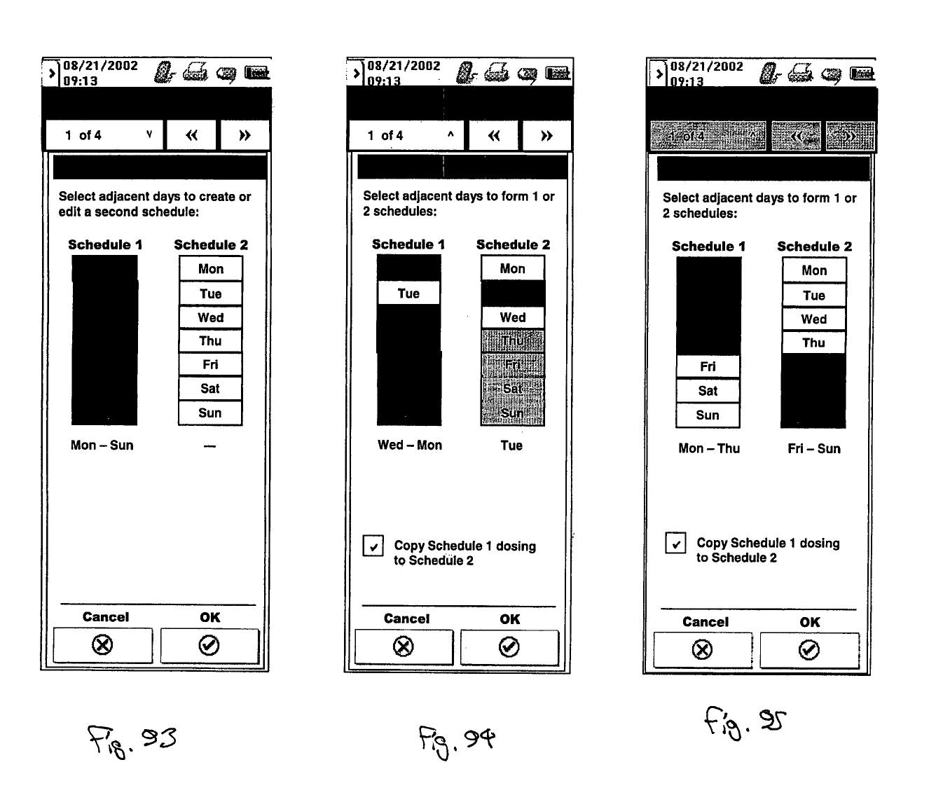

FIGS. 93 through 95 illustrate screen shots in which a programmed drug infusion schedule for one day, or a group of days, may be copied to another day or group of days. The ability to copy a drug infusion program created for one day to another day or multiple days can greatly the ease in creating a multiple step drug infusion program. In FIG. 93, a first schedule, schedule 1, is shown on the left and a second schedule, schedule 2, is shown on the right. In FIG. 94, Tuesday is selected from schedule 1 on the left and Tuesday is selected from schedule 2 on the right. A check is entered in the box confirming that drug infusion program for Tuesday of schedule 1 is being copied to Tuesday of schedule 2. In FIG. 95, a group of days in schedule 1, namely Monday-Thursday is being copied as a group.

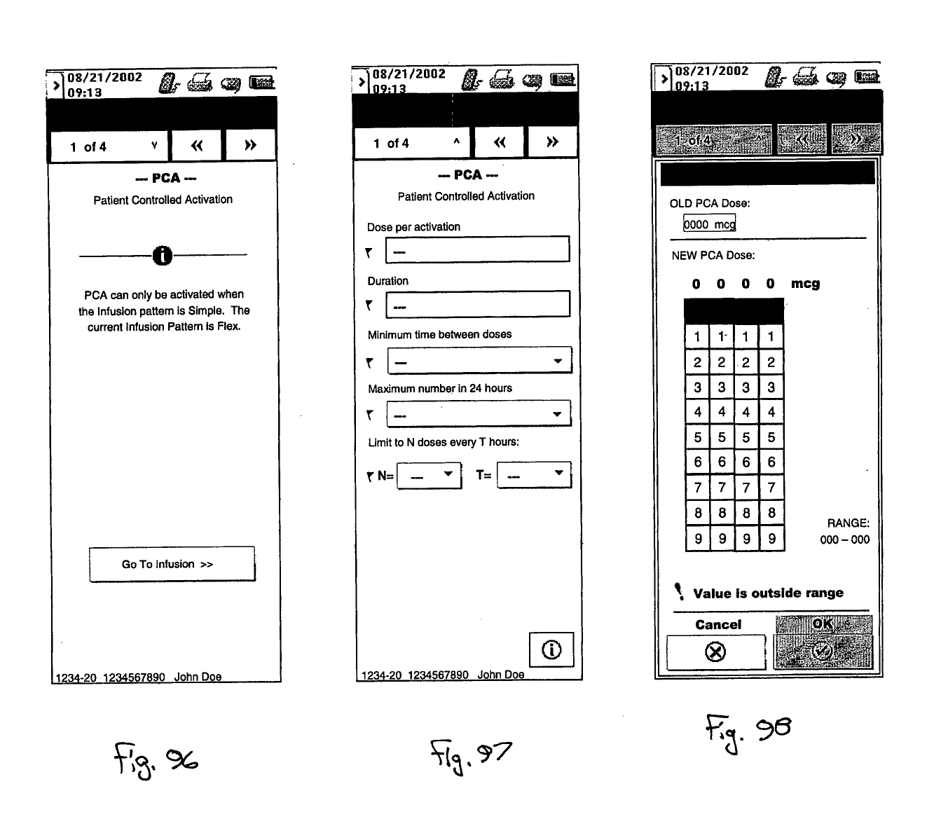

FIGS. 96-98 illustrate screen shots showing implementation of the patient controlled analgesia (PCA) task area. These screens may be reached by selecting, e.g., by tapping, the arrows associated with the PCA task area in any of the initial status screens. In FIG. 96, a warning is provided that the patient controlled analgesia drug infusion program can only be implemented on top the simple drug infusion program or when the infusion program is constant. If the infusion is not constant, the medical professional is invited to change the infusion pattern.

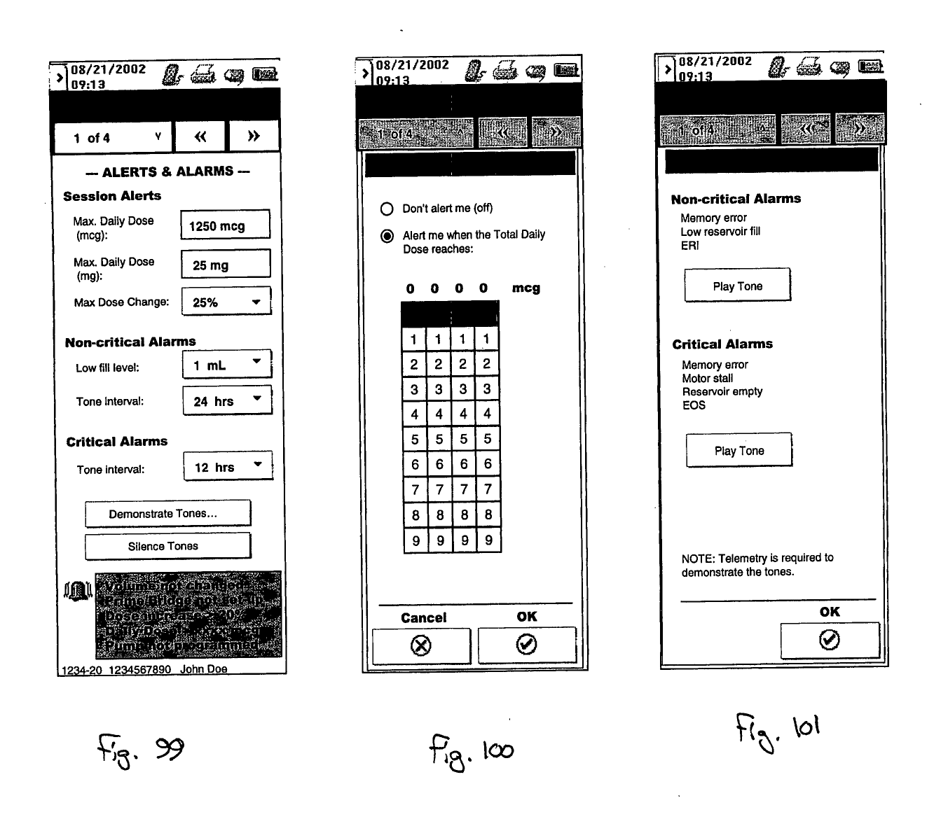

In FIG. 97, detailed information about the patient controlled activation may be provided including the dose per activation, the duration of each activation, the minimum time between activations and the maximum number of activations per unit time period. The latter may be specified as being limited to N doses every T hours. Thus, the unit time period does not need to be twenty-four hours. In FIG. 98, a screen is provided allowing input of a new dosage amount while maintaining display of the previous dosage amount. A warning may be provided if the value entered is outside of a predetermined range or value.

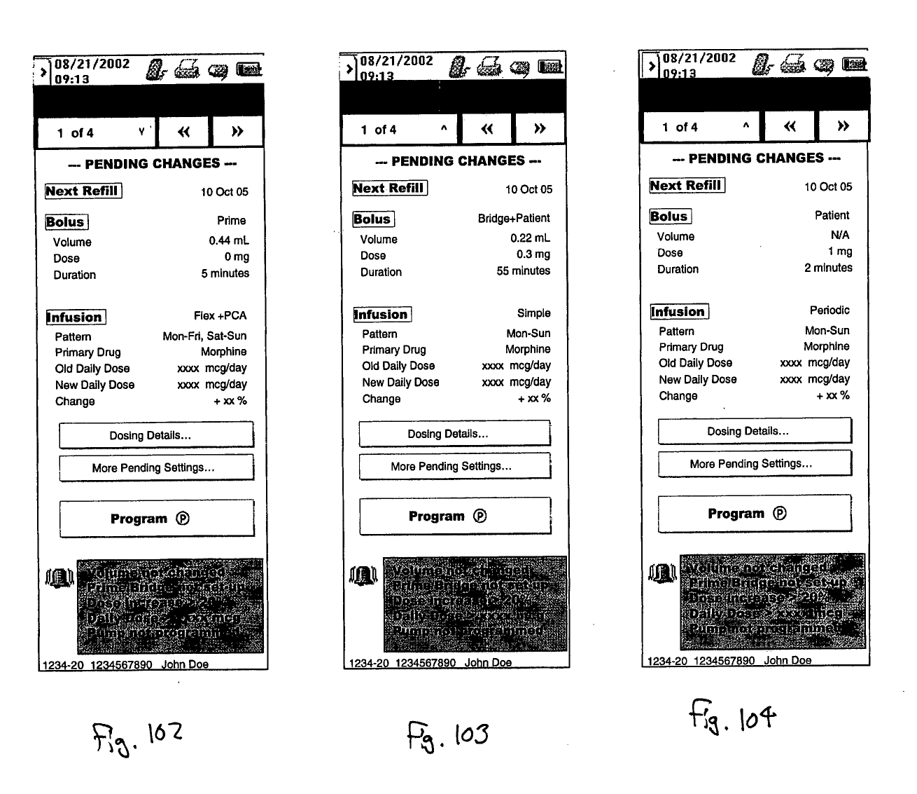

FIGS. 99-101 illustrate screen shots showing implementation of the alarm task area. These screens may be reached by selecting, e.g., by tapping, the arrows associated with the alarm task area in any of the initial status screens. FIG. 99 shows a screen that allows the medical professional to set session alerts. For example, the medical professional may set limits, for a maximum total daily dose, a maximum concentration change, a maximum dose change and a low fill level. The interval for alarm tones may be selected. FIG. 100 illustrates the interface for actually entering the maximum dosage amount, e.g., total daily dose, listed in FIG. 99. FIG. 101 illustrates an interface for which alarms are to be considered non-critical and which alarms are to be considered critical as well as tones to be played for each.

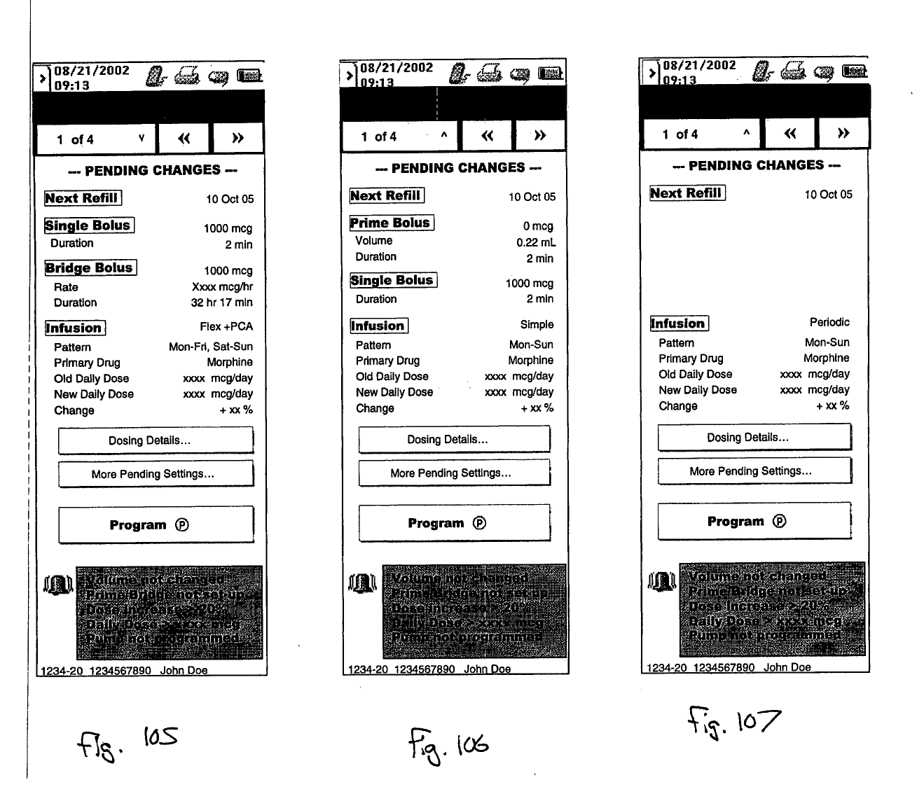

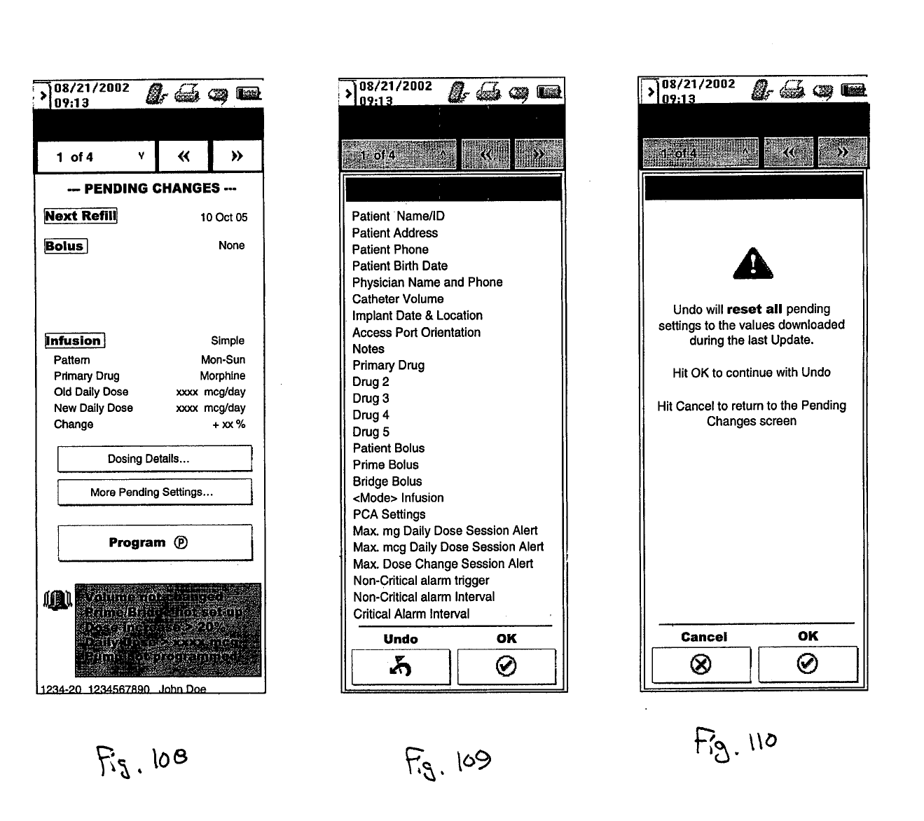

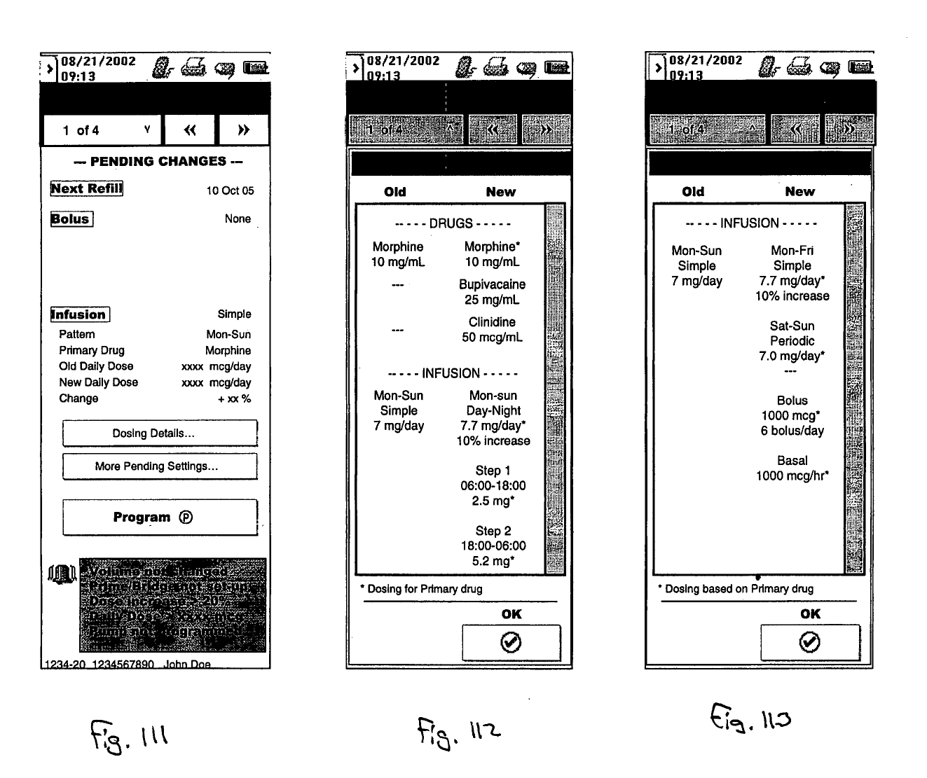

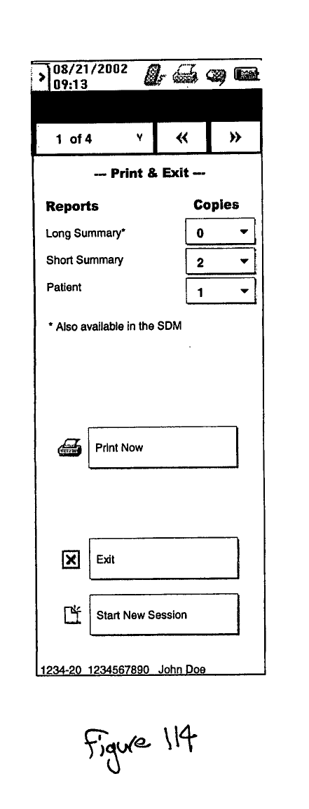

FIGS. 102 through 113 illustrate screen shots associated with changes made by the medical professional in the preceding tasks. This summary can be useful to the medical professional to confirm what has been accomplished on the other screens. This information is only a summary of the changes made in controller 20. The changes have not yet been downloaded to the implanted medical device. Hence, the medical professional may still review and modify the programming accomplished to this point. FIGS. 102 through 108 provide alternative implementations of pending changes screen shots depending, for example, on the type of drug infusion program programming into controller 20. FIG. 109 provides a summary all program changes to be downloaded and entered into implantable drug infusion device 14. FIG. 110 provides one last chance to undo the contemplated changes before the pending values are downloaded to the implantable drug infusion device 14. FIG. 111 illustrates a screen shot summarizing the changed information downloaded to implantable drug infusion device 14. FIGS. 113 and 114 provide a summary of the changed information with the old information provided in the left column and the changed information provided in the right column.

FIG. 114 illustrates a screen shot providing for the printing of reports such as a long summary, a short summary and/or patient information. The medical professional may print, e.g., to a hard copy or to a file or disk or message, a report of the information programmed in controller 20. Thus, the program information may be retained for future reference.

Once the program information is complete, the programmed information that is necessary for the implanted medical device to operate, and any other desired, can then be sent, for example, by telemetry to the implanted medical device and the new programmed amounts and features become effective.

In one embodiment, a system is capable of delivering a therapeutic output to a patient. An implantable medical device is capable of delivering the therapeutic output to the patient. A controller, programmable by a medical professional, is operatively coupled to the implantable medical device, to specify, at least in part, the therapeutic output to be delivered to the patient. The controller is operable to specify the therapeutic output through specification of a plurality of tasks. The controller has an interface with the medical professional in order to accomplish at least one of a plurality of procedures, each of the plurality of procedures including at least some of the plurality of tasks. The controller is selectable by the medical professional to perform one of the plurality of procedures. The controller presents the interface with the at least some of the plurality of tasks to be performed by the medical professional based upon a selected one of the plurality of procedures.

In an embodiment, the interface only includes tasks to be performed by the medical professional that are associated with the selected one of the plurality of procedures.

In an embodiment, the interface presents the tasks in a chronological order of implementation by the medical professional.

In an embodiment, the interface hides tasks not associated with the selected one of the plurality of procedures.

In an embodiment, the interface also provides an option to the medical professional to select any of the plurality of tasks following selection of the selected one of the plurality of procedures.

In an embodiment, an otherwise hidden task selected under the option again is again hidden when the medical professional returns to the selected one of the plurality of procedures.

In another embodiment, a controller for an implantable medical device is capable of delivering a therapeutic output to a patient. A control module, operatively coupled to the implantable medical device, is programmable by a medical professional to specify, at least in part, the therapeutic output to be delivered to the patient. The control module is selectable by the medical professional to perform one of the plurality of procedures. The control module presents the interface with the at least some of the plurality of tasks to be performed by the medical professional based upon a selected one of the plurality of procedures.

In an embodiment, the interface only includes tasks to be performed by the medical professional that are associated with the selected one of the plurality of procedures.

In an embodiment, the interface presents the tasks in a chronological order of implementation by the medical professional.

In an embodiment, the interface hides tasks not associated with the selected one of the plurality of procedures.

In an embodiment, the interface also provides an option to the medical professional to select any of the plurality of tasks following selection of the selected one of the plurality of procedures.

In an embodiment, an otherwise hidden task selected under the option again is again hidden when the medical professional returns to the selected one of the plurality of procedures.

In another embodiment, a method controls an implantable medical device capable of delivering a therapeutic output to a patient, the implantable medical device being programmable by a medical professional to specify through a series of tasks, at least in part, the therapeutic output to be delivered to the patient. An interface is presented to the medical professional for selection of one of a plurality of procedures to be performed in controlling the implantable medical device. An interface is presented, based at least in part on the selection of one of a plurality of procedures, to the medical professional of at least some of the tasks to be performed by the medical professional. The tasks are performed by the medical professional.

In an embodiment, the interface only includes tasks to be performed by the medical professional that are associated with the selected one of the plurality of procedures.

In an embodiment, the interface presents the tasks in a chronological order of implementation by the medical professional.

In an embodiment, the interface hides tasks not associated with the selected one of the plurality of procedures.

In an embodiment, the interface also provides an option to the medical professional to select any of the plurality of tasks following selection of the selected one of the plurality of procedures.

In another embodiment, a system is capable of delivering a therapeutic output to a patient. An implantable medical device is capable of delivering the therapeutic output to the patient. A controller, programmable by a medical professional, is operatively coupled to the implantable medical device, to specify, at least in part, the therapeutic output to be delivered to the patient. The controller is operable to specify the therapeutic output through specification of a plurality of tasks. The controller has an interface providing a first screen presenting the medical professional with at least some of the plurality of tasks on the first screen with the first screen divided into a plurality of task areas with each of the at least some of the plurality of tasks associated with a different one of the plurality of task areas.

In an embodiment, the interface presents a second screen associated with a particular one of the plurality of tasks upon selection of the task by the medical professional from the first screen.

In an embodiment, the interface represents the first screen upon completion by the medical professional of the particular one of the plurality of tasks.

In an embodiment, the first screen distinctly identifies the tasks already selected by the medical professional.

In another embodiment, a controller for an implantable medical device is capable of delivering a therapeutic output to a patient. A control module, operatively coupled to the implantable medical device, is programmable by a medical professional to specify, at least in part, the therapeutic output to be delivered to the patient. The control module is operable to specify the therapeutic output through specification of a plurality of tasks. The control module has an interface providing a first screen presenting the medical professional with at least some of the plurality of tasks on the first screen with the first screen divided into a plurality of task areas with each of the at least some of the plurality of tasks associated with a different one of the plurality of task areas.

In an embodiment, the interface presents a second screen associated with a particular one of the plurality of tasks upon selection of the task by the medical professional from the first screen.

In an embodiment, the interface represents the first screen upon completion by the medical professional of the particular one of the plurality of tasks.

In an embodiment, the first screen distinctly identifies the tasks already selected by the medical professional.

In another embodiment, a method controls an implantable medical device capable of delivering a therapeutic output to a patient, the implantable medical device being programmable by a medical professional to specify through a plurality of tasks, at least in part, the therapeutic output to be delivered to the patient. An interface is presented providing a first screen presenting the medical professional with at least some of the plurality of tasks on the first screen with the first screen divided into a plurality of task areas with each of the at least some of the plurality of tasks associated with a different one of the plurality of task areas. A second screen is presented associated with a particular one of the plurality of tasks upon selection of the task by the medical professional from the first screen.

In an embodiment, the interface represents the first screen upon completion by the medical professional of the particular one of the plurality of tasks.

In an embodiment, the first screen distinctly identifies the tasks already selected by the medical professional.

In another embodiment, a system is capable of delivering a therapeutic output to a patient. An implantable medical device is capable of delivering the therapeutic output to the patient. A controller, programmable by a medical professional, is operatively coupled to the implantable medical device, to specify, at least in part, the therapeutic output to be delivered to the patient in a series of discrete time intervals over a time period. The controller has an interface allowing the medical professional to graphically select an amount of the therapeutic output to be delivered to the patient in at least one of the series of discrete timer intervals.

In an embodiment, the interface of the controller provides a graphical display of the amount of the therapeutic output in each of the series of discrete time intervals over at least a part of the time period.

In an embodiment, the interface of the controller allows the medical professional to graphically modify the amount of the therapeutic output to be delivered to the patient by graphically dragging a portion of the graphical display associated with at least a particular one of the series of discrete time intervals.

In an embodiment, the interface of the controller graphically displays the amount of the therapeutic output to be delivered to the patient in each of the series of discrete time intervals over all of the time period.

In another embodiment, a controller for an implantable medical device is capable of delivering a therapeutic output to a patient. A control module, operatively coupled to the implantable medical device, is programmable by a medical professional to specify, at least in part, the therapeutic output to be delivered to the patient in a series of discrete time intervals over a time period. The controller has an interface allowing the medical professional to graphically select an amount of the therapeutic output to be delivered to the patient in at least one of the series of discrete timer intervals.

In an embodiment, the interface of the controller provides a graphical display of the amount of the therapeutic output in each of the series of discrete time intervals over at least a part of the time period.

In an embodiment, the interface of the controller allows the medical professional to graphically modify the amount of the therapeutic output to be delivered to the patient by graphically dragging a portion of the graphical display associated with at least a particular one of the series of discrete time intervals.

In an embodiment, the interface of the controller graphically displays the amount of the therapeutic output to be delivered to the patient in each of the series of discrete time intervals over all of the time period.

In another embodiment, a method controls an implantable medical device capable of delivering a therapeutic output to a patient, the implantable medical device being programmable by a medical professional to specify, at least in part, the therapeutic output to be delivered to the patient in a series of discrete time intervals over a time period. An interface presents a graphical depiction of amount of the therapeutic output to be delivered to the patient over the series of discrete time intervals. The medical professional may graphically select an amount of the therapeutic output to be delivered to the patient in at least one of the series of discrete timer intervals.

In an embodiment, the interface of the controller provides a graphical display of the amount of the therapeutic output in each of the series of discrete time intervals over at least a part of the time period.

In an embodiment, the interface of the controller allows the medical professional to graphically modify the amount of the therapeutic output to be delivered to the patient by graphically dragging a portion of the graphical display associated with at least a particular one of the series of discrete time intervals.

In an embodiment, the interface of the controller graphically displays the amount of the therapeutic output to be delivered to the patient in each of the series of discrete time intervals over all of the time period.

In another embodiment, a system is capable of delivering a therapeutic output to a patient. An implantable medical device is capable of delivering the therapeutic output to the patient. A controller, programmable by a medical professional, is operatively coupled to the implantable medical device, to specify, at least in part, the therapeutic output to be delivered to the patient. The controller is operable to specify the therapeutic output through specification of a plurality of tasks and having an interface with the medical professional. The interface accomplishes at least one the plurality of tasks through a series of questions and responses.

In an embodiment, the series of questions and responses are presented in clinical terms rather than engineering terms.

In an embodiment, the questions in the series of questions and responses are conversational.

In an embodiment, the controller further provides explanations in a conversational language.

In an embodiment, the controller performs calculations in response to the series of questions and responses in order to properly program the implantable medical device and wherein the controller presents a worksheet illustrating the calculations to the medical professional.

In an embodiment, the controller displays to the medical professional a percentage change of the therapeutic output as a result of any changes made by the medical professional.

In an embodiment, the controller displays the percentage change before making any such changes effective in the implantable medical device.

In another embodiment, a controller for an implantable medical device is capable of delivering a therapeutic output to a patient. A control module, programmable by a medical professional, is operatively coupled to the implantable medical device to specify, at least in part, the therapeutic output to be delivered to the patient. The controller is operable to specify the therapeutic output through specification of a plurality of tasks and having an interface with the medical professional. The interface accomplishes at least one the plurality of tasks through a series of questions and responses.

In an embodiment, the series of questions and responses are presented in clinical terms rather than engineering terms.

In an embodiment, the questions in the series of questions and responses are conversational.

In an embodiment, the controller further provides explanations in a conversational language.

In another embodiment, the controller of an implantable medical device performs calculations in response to the series of questions and responses in order to properly program the implantable medical device and wherein the controller presents a worksheet illustrating the calculations to the medical professional.

In an embodiment, the controller displays to the medical professional a percentage change of the therapeutic output as a result of any changes made by the medical professional.

In an embodiment, the controller displays the percentage change before making any such changes effective in the implantable medical device.

The contents of U.S. Provisional Patent Application Ser. No. 60/602,489, Wende L. Dewing, Dale R. Ulbrich and Pavankumar Dadlani, filed Aug. 18, 2004, entitled Interface For Programmable Implantable Medical Device, is hereby incorporated by reference in its entirety.

Thus, embodiments of the invention are disclosed. One skilled in the art will appreciate that the present invention can be practiced with embodiments other than those disclosed.

The disclosed embodiments are presented for purposes of illustration and not limitation, and the present invention is limited only by the claims that follow.

What is claimed is:

1. A system capable of delivering a therapeutic output to a patient, comprising:
an implantable medical device capable of delivering said therapeutic output to said patient;
a controller, programmable by a medical professional, operatively coupled to said implantable medical device, to specify, at least in part, said therapeutic output to be delivered to said patient;
said controller being operable to specify said therapeutic output through specification of a plurality of tasks; and
said controller having an interface providing a first screen presenting said medical professional with at least some of said plurality of tasks on said first screen with said first screen divided into a plurality of task areas with each of said at least some of said plurality of tasks associated with a different one of said plurality of task areas.

2. A system as in claim 1 wherein said interface presents a second screen associated with a particular one of said plurality of tasks upon selection of said task by said medical professional from said first screen.

3. A system as in claim 2 wherein said interface represents said first screen upon completion by said medical professional of said particular one of said plurality of tasks.

4. A system as in claim 3 wherein said first screen distinctly identifies said tasks already selected by said medical professional.

5. A system as in claim 4 wherein said first screen distinctly identifies said tasks already completed by said medical professional.

6. A system as in claim 1 wherein said interface allows said medical professional to graphically select an amount of said therapeutic output to be delivered to said patient in at least one of said series of discrete timer intervals.

7. A system as in claim 1 wherein said interface accomplishes at least one said plurality of tasks through a series of questions and responses.

8. A controller for an implantable medical device capable of delivering a therapeutic output to a patient, comprising:
a control module, operatively coupled to said implantable medical device, being programmable by a medical professional to specify, at least in part, said therapeutic output to be delivered to said patient;
said control module being operable to specify said therapeutic output through specification of a plurality of tasks; and
said control module having an interface providing a first screen presenting said medical professional with at least some of said plurality of tasks on said first screen with said first screen divided into a plurality of task areas with each of said at least some of said plurality of tasks associated with a different one of said plurality of task areas.

9. A controller as in claim 8 wherein said first screen distinctly identifies said tasks already selected by said medical professional.

10. A controller as in claim 8 wherein said interface presents a second screen associated with a particular one of said plurality of tasks upon selection of said task by said medical professional from said first screen.

11. A controller as in claim 10 wherein said interface represents said first screen upon completion by said medical professional of said particular one of said plurality of tasks.

12. A controller as in claim 11 wherein said first screen distinctly identifies said tasks already selected by said medical professional.

13. A controller as in claim 8 wherein interface allows said medical professional to graphically select an amount of said therapeutic output to be delivered to said patient in at least one of said series of discrete timer intervals.

14. A controller as in claim 8 wherein said interface accomplishes at least one said plurality of tasks through a series of questions and responses.

15. A method of controlling an implantable medical device capable of delivering a therapeutic output to a patient, said implantable medical device being programmable by a medical professional to specify through a plurality of tasks, at least in part, said therapeutic output to be delivered to said patient, comprising the steps of:
presenting an interface providing a first screen presenting said medical professional with at least some of said plurality of tasks on said first screen with said first screen divided into a plurality of task areas with each of said at least some of said plurality of tasks associated with a different one of said plurality of task areas; and
presenting a second screen associated with a particular one of said plurality of tasks upon selection of said task by said medical professional from said first screen.

16. A method as in claim 15 wherein said first screen distinctly identifies said tasks already selected by said medical professional.

17. A method as in claim 15 wherein said first screen distinctly identifies said tasks already completed by said medical professional.

18. A method as in claim 15 wherein said interface represents said first screen upon completion by said medical professional of said particular one of said plurality of tasks.

19. A method as in claim 18 wherein said first screen distinctly identifies said tasks already completed by said medical professional.

20. A method as in claim 18 wherein said first screen distinctly identifies said tasks already selected by said medical professional.

21. A computer readable medium for controlling an implantable medical device capable of delivering a therapeutic output to a patient, said implantable medical device being programmable by a medical professional to specify through a plurality of tasks, at least in part, said therapeutic output to be delivered to said patient, comprising:
presenting an interface providing a first screen presenting said medical professional with at least some of said plurality of tasks on said first screen with said first screen divided into a plurality of task areas with each of said at least some of said plurality of tasks associated with a different one of said plurality of task areas; and
presenting a second screen associated with a particular one of said plurality of tasks upon selection of said task by said medical professional from said first screen.
